(19) United States
(12) Patent Application Publication
(10) Pub. No.: US 2022/0117514 A1
(43) Pub. Date: Apr. 21, 2022

(54) SYSTEM AND METHOD FOR DETERMINING QUANTITATIVE HEALTH-RELATED PERFORMANCE STATUS OF A PATIENT

(71) Applicant: University of Southern California, Los Angles, CA (US)

(72) Inventors: Peter Kuhn, Solana Beach, CA (US); Jorge Javier Nieva, Pasadena, CA (US); Luciano Pasquale Nocera, Los Angeles, CA (US)

(21) Appl. No.: 17/433,212

(22) PCT Filed: Mar. 27, 2020
(86) PCT No.: PCT/US2020/025536

§ 371 Date: Aug. 23, 2021

Related U.S. Application Data
(60) Provisional application No. 62/825,965, filed on Mar. 29, 2019.

(51) Int. Cl. A61B 5/11; A61B 5/00

(52) U.S. Cl. CPC A61B 5/1118; A61B 5/1128; A61B 5/1121; A61B 5/1116; A61B 5/1113; A61B 5/681; A61B 5/0022; A61B 5/4836; A61B 5/743; A61B 5/7246; A61B 5/7267; A61B 5/7275; A61B 5/6898

(57) ABSTRACT

This disclosure relates to a system for determining a quantitative health-related performance status of a patient. This disclosure further relates to a health assessment method for quantitative determination of health-related performance or quality of life of a patient. More specifically, this disclosure relates to systems and methods for determining whether a cancer patient will need unplanned medical care during cancer therapy. This system may comprise at least one sensor and at least one processor. The system may be configured to generate at least one output signal conveying physical activity information corresponding to physical activity of the patient, or spatial position information corresponding to at least one spatial position of an anatomical site of the patient while the patient performs a movement. The system may further be configured to determine a quantitative health-related performance score of the patient based on the physical activity parameter or the kinematic parameter. The system may further be configured to determine whether the patient will need unplanned medical care during a therapy based on the quantitative health-related performance score. The movement performed by the patient may be a prescribed movement.

### CROSS REFERENCE TO RELATED APPLICATIONS

This application claims priority under 35 U.S.C. § 119 to U.S. Provisional Application No. 62/825,965, filed Mar. 29, 2019, the disclosures of which are incorporated herein by reference in their entirety.

### FIELD OF THE DISCLOSURE

This disclosure also relates to a system for determining a quantitative health-related performance status of a patient. This disclosure further relates to a quantitative health assessment method for quantitative determination of health-related performance or quality of life of a patient. More specifically, this disclosure relates to systems and methods for determining whether a cancer patient will need unplanned medical care during cancer therapy.

### BACKGROUND

Biomechanical characterization of human performance is known. Using biomechanical characterization of human performance to inform decisions about oncological therapy in an effort to reduce or avoid a need for unplanned medical care (e.g., caused by deterioration of a cancer patient) is also known. However, typical biomechanical characterization of human performance for oncological or other reasons often comprises either a qualitative assessment by medical personnel, or an invasive biomechanical characterization test. These require significant experimental setup that includes numerous sensors. In addition, qualitative assessments are difficult to standardize due to their intrinsically subjective nature. Invasive tests provide reliable information but are not feasible for large scale applications.

How patients move in the office provides clinicians with valuable information about frailty. This is particularly important for patients undergoing arduous treatments such as chemotherapy. When describing these metrics, the physician assessment is often qualitative, subjective, and lacks agreement among observers. Quantitative imaging tools have the potential to provide an objective and verifiable measurement of physician observations of patients in the office.

Each patient has specific and individual needs for optimal supportive care during cancer treatment. Predicting these needs and providing specific solutions has the opportunity to both improve outcomes and the experience during treatment. Poor patient outcomes, patient satisfaction, quality of life, and economic cost are associated with unexpected hospitalizations with patients actively receiving chemotherapy. A recent survey of US oncology nurses found that 61% of nurses cared for patients who had to go to the emergency room or were hospitalized due to chemotherapy induced nausea and vomiting (CINV). These CINV hospitalization costs were estimated to be over $15,000 per occurrence. Readily available tools and metrics such as ECOG performance status, Body Mass Index (BMI), Mini Mental State Exam (MMSE), and Charlson Comorbidity Index (CCI), are part of a comprehensive geriatric assessment, however few physicians perform the complete assessment, as they are time consuming. There is emerging data that a comprehensive geriatric assessment can predict complications and side effects from treatment.

Currently, the most routine assessment is the ECOG performance status. It is well known that in metastatic cancer such as lung origin, ECOG strongly predicts survival independent of treatment and usually guides if treatment should even be given if poor performance status. Clinical assessment of performance status and risk of toxicity from cancer therapy includes observation of patient movement as part of the physician examination within a clinic room environment. This has been routine practice for many years, and while it has been recognized for a long time, oncologists and patients substantially differ in their assessment of performance status with most oncologists being overly optimistic on the patient's performance status.

The utility of activity trackers has been evaluated in areas outside of cancer medicine and demonstrated correlation with clinical outcomes in a wide variety of other disease settings. For example, in COPD, increasing additional steps correlates with reduced COPD hospitalizations and formal exercise capacity evaluation such as the six-minute walk distance predicted COPD-related hospitalization. After cardiac surgery, it was observed using an accelerometer that inpatient step count appears to predict repeat hospitalization. In elderly hospitalizations it was found that mobility after hospital discharge could predict 30-day hospital readmissions.

To improve our understanding unexpected hospital visits in cancer patients receiving chemotherapy we conducted an observational study to evaluate the effect of physical activity as measured by a motion-capture system and wearable movement sensor and their relationship to unexpected healthcare encounters.

### REFERENCES

The following publications are to assist in understanding the disclosure.
Roeland, E, Ma J, Binder G, Goldberg R, Paglia R, Knoth R, Schwartzberg L. Hospitalization costs for nausea and vomiting: a savings opportunity. Journal of Clinical Oncology 2017, 35:31 suppl, 155-155.
Clark-Snow R, Affronti M L, Rittenberg C N. Chemotherapy-induced nausea and vomiting (CINV) and adherence to antiemetic guidelines: results of a survey of oncology nurses. Supportive Care in Cancer 2018; 26(2):557-564.
Freyer G, Geay J F, Touzet S, et al. Comprehensive geriatric assessment predicts tolerance to chemotherapy and survival in elderly patients with advanced ovarian carcinoma: a GINECO study. Ann Oncol 2005; 16:1795.
Extermann M, Boler I, Reich R R, et al. Predicting the risk of chemotherapy toxicity in older patients: the Chemotherapy Risk Assessment Scale for High-Age Patients (CRASH) score. Cancer 2012; 118:3377.
Hurria A, Togawa K, Mohile S G, et al. Predicting chemotherapy toxicity in older adults with cancer: a prospective multicenter study. J Clin Oncol 2011; 29:3457.
Extermann M, Bonetti M, Sledge G W, et al. MAX2—a convenient index to estimate the average per patient risk for chemotherapy toxicity; validation in ECOG trials. Eur J Cancer 2004; 40:1193.
Ramjaun A, Nassif M O, Krotneva S, et al. Improved targeting of cancer care for older patients: a systematic review of the utility of comprehensive geriatric assessment. J Geriatr Oncol 2013; 4:271.
Hamaker M E, Prins M C, Stauder R. The relevance of a geriatric assessment for elderly patients with a haematological malignancy—a systematic review. Leuk Res 2014; 38:275.
Corre R, Greillier L, Le Caer H, et al. Use of a Comprehensive Geriatric Assessment for the Management of Elderly Patients With Advanced Non-Small-Cell Lung Cancer: The Phase III Randomized ESOGIA-GFPC-GECP 08-02 Study. J Clin Oncol 2016; 34:1476.
Karnofsky, D. A., R. R. Ellison, and R. B. Golbey, Selection of patients for evaluation of chemotherapeutic procedures in advanced cancer. J Chronic Dis, 1962; 15: p. 243-9.
Gridelli, C. and J. Hainsworth, Meeting the chemotherapy needs of elderly and poor performance status patients with NSCLC. Lung Cancer, 2002; 38 Suppl 4: p. 37-41.
Hainsworth, J. D., et al., Weekly combination chemotherapy with docetaxel and gemcitabine as first-line treatment for elderly patients and patients with poor performance status who have extensive-stage small cell lung carcinoma: a Minnie Pearl Cancer Research Network phase II trial. Cancer 2004; 100(11): p. 2437-41.
Lee, K. W., et al., Weekly low-dose docetaxel for salvage chemotherapy in pretreated elderly or poor performance status patients with non-small cell lung cancer. J Korean Med Sci 2008; 23(6): p. 992-8.
Sweeney, C. J., et al., Outcome of patients with a performance status of 2 in Eastern Cooperative Oncology Group Study E1594: a Phase II trial in patients with metastatic nonsmall cell lung carcinoma. Cancer 2001; 92(10): p. 2639-47.
Ou, S. H. and J. A. Zell, Validation study of the proposed IASLC staging revisions of the T4 and M non-small cell lung cancer descriptors using data from 23,583 patients in the California Cancer Registry. J Thorac Oncol 2008; 3(3): p. 216-27.
Ando M, Ando Y, Hasegawa Y, et al. Prognostic value of performance status assessed by patients themselves, nurses, and oncologists in advanced non-small cell lung cancer. British Journal of Cancer. 2001; 85(11):1634-1639. doi:10.1054/bjoc.2001.2162.
Taylor, A. E., et al., Observer error in grading performance status in cancer patients. Supportive Care in Cancer 1999; 7(5): p. 332-335.
Nguyen, M. N. B., et al., “Mining Human Mobility to Quantify Performance Status,” 2017 IEEE International Conference on Data Mining Workshops (ICDMW), New Orleans, L A, 2017, pp. 1172-1177. doi: 10.1109/ICDMW.2017.168
Cook, D., et al. Functional recovery in the elderly after major surgery: Assessment of mobility recovery using wireless technology. Ann Thorac Surg 2013; 96: 1057-61.
Donaire-Gonzalez, D., et al. Benefits of physical activity on COPD hospitalization depend on intensity. European Respiratory Journal 2014; 46(5) 1281-1289.
Durheim, M, et al. Six-minute-walk distance and accelerometry predict outcomes in chronic obstructive pulmonary disease independent of global initial for chronic obstructive lung disease 2011 group. American Thoracic Society 2015; 12(3): 349-356.
Takahashi, T., et al. In-patient step count predicts re-hospitalization after cardiac surgery. J Cardiology 2014; 66: 286-191.
Fisher, S, et al. Mobility after hospital discharge as a marker for 30-day readmission. Journal of Gerontology 2013; 68(7): 805-810.
Butland, R. J., et al., Two-, six-, and 12-minute walking tests in respiratory disease. Br Med J (Clin Res Ed) 1982; 284(6329): p. 1607-8.
Zaki Hasnain, Ming Li, Tanya Dorff, David Quinn, Naoto T. Ueno, Sriram Yennu, Anand Kolatkar, Cyrus Shahabi, Luciano Nocera, Jorge Nieva, Peter Kuhn, Paul K. Newton, Low-dimensional dynamical characterization of human performance of cancer patients using motion data, Clinical Biomechanics 2018; 56:61-69.
Alexander S. Martin, Roger Wilson Boles, Luciano Nocera, Anand Kolatkar, Marcella May, Zaki Hasnain, Naoto T. Ueno, Sriram Yennu, Angela Alexander, Aaron Mejia, Ming Li, Frankie A. Cozzens Philips, Paul K. Newton, Joan Broderick, Cyrus Shahabi, Peter Kuhn, Jorge J. Nieva. Objective metrics of patient activity: Use of wearable trackers and patient reported outcomes in predicting unexpected healthcare events in cancer patients undergoing highly emetogenic chemotherapy. J Clin Oncol 36, 2018 (suppl; abstr 6519).
Nail, L. M., My get up and go got up and went: fatigue in people with cancer. J Natl Cancer Inst Monogr, 2004; 32:72-5.
Wall, J. C., et al., The Timed Get-up-and-Go test revisited: measurement of the component tasks. J Rehabil Res Dev 2000; 37(1): p. 109-13.
Ruxton, G. D. (2006). The unequal variance t-test is an underused alternative to Student's t-test and the Mann-Whitney U test. Behavioral Ecology 2006; 17(4), 688-690.
Brewer W, Swanson B T, Ortiz A. Validity of Fitbit's active minutes as compared with a research-grade accelerometer and self-reported measures. BMJ Open Sport &amp; Exercise Medicine 2017; 3(1).
Gupta A, Stewart T, Bhulani N, Dong Y, Rahimi Z, Crane K, et al. Feasibility of Wearable Physical Activity Monitors in Patients With Cancer. JCO Clinical Cancer Informatics 2018; (2):1-10.
Pirl W F, Fujisawa D, Stagl J, Eusebio J, Traeger L, El-Jawahri A, et al. Actigraphy as an objective measure of performance status in patients with advanced cancer. Journal of Clinical Oncology 2015; 33(29_suppl):62-.
Suh S-Y, LeBlanc T W, Shelby R A, Samsa G P, Abernethy A P. Longitudinal Patient-Reported Performance Status Assessment in the Cancer Clinic Is Feasible and Prognostic. Journal of Oncology Practice 2011; 7(6):374-81.
Popovic G, Pope A, Harhara T, Swami N, Le L, Zimmermann C. Agreement between physician and patient performance status ratings in an outpatient setting. Journal of Clinical Oncology 2015; 33(29_suppl):66-.
Walsh J, Hussey J, O'Donnell D. A pilot study comparing objective physical activity to the physical component of the Eastern Cooperative Oncology Group (ECOG) performance status scale. Journal of Clinical Oncology 2009; 27(15S):e20501-e.
Burke T A, Wisniewski T, Ernst F R. Resource utilization and costs associated with chemotherapy-induced nausea and vomiting (CINV) following highly or moderately emetogenic chemotherapy administered in the US outpatient hospital setting. Support Care Cancer 2011; 19:131-140.
Handley N, Schuchter L, Bekelman J. Best Practices for Reducing Unplanned Acute Care for Patients with Cancer. Journal of Oncology Practice 2018; 14:5, 306-313.
Cheng S, Qureshi M, Pullenayegum E, Haynes A, Chan K K. Do patients with reduced or excellent performance status derive the same clinical benefit from novel systemic cancer therapies? A systematic review and meta-analysis. ESMO Open 2017; 2(4).

### SUMMARY

This disclosure relates to a system for determining a quantitative health-related performance status of a patient. This system may comprise at least one sensor, and at least one processor. The system may be configured to generate at least one output signal conveying physical activity information corresponding to physical activity of the patient, or spatial position information corresponding to at least one spatial position of an anatomical site of the patient while the patient performs a movement. The system may further be configured to determine at least one physical activity parameter or at least one kinematic parameter based on the at least one output signal. The system may further be configured to determine a quantitative health-related performance score of the patient based on the physical activity parameter or the kinematic parameter. The system may further be configured to determine whether the patient will need unplanned medical care during a therapy based on the quantitative health-related performance score.

In this disclosure, the movement performed by the patient may be a prescribed movement. The prescribed movement may comprise movement associated with a chair to table (CTT) exam and/or a get up and walk (GUP) exam.

In this disclosure, the system may further comprise an information conveying device that conveys information to a human user. The conveyed information may be related to the quantitative health-related performance score and/or the determination of whether the patient will need unplanned medical care. In this disclosure, the information conveying device may be configured to convey information by sound, a text, an image, a mechanical action, the like, or a combination thereof. The at least one sensor may generate the at least one output signal conveying physical activity information corresponding to physical activity of the patient, or the spatial position information corresponding to at least one spatial position of an anatomical site of the patient while the patient performs a movement. The at least one sensor may comprise a body position sensor and/or a physical activity sensor.

In this disclosure, the system may further comprise a system comprising an image recording device. The system may further comprise a system comprising a 3D motion capture device. The system may further comprise a system comprising a 3D motion capture device. The 3D motion capture device may comprise an image recording device, a time-of-flight measurement device, a heat sensor, the like, and a combination thereof. The system may further comprise a system comprising a ToF sensor.

In this disclosure, the at least one processor determines the at least one physical activity parameter or at least one kinematic parameter based on the at least one output signal. The at least one processor determines the quantitative health-related performance score of the patient based on the physical activity parameter or the kinematic parameter. In this disclosure, the at least one processor determines whether the patient will need unplanned medical care during a therapy based on the quantitative health-related performance score. The at least one sensor may comprise a body position sensor, a wearable physical activity tracker, a balance, a system comprising an image recording device, a display, or a combination thereof. The at least one sensor may comprise a wrist worn motion sensor. The system may comprise a mobile phone.

In this disclosure, the anatomical site comprises the patient's body or the patient's body part. In this disclosure, the anatomical site comprises a center of mass of the patient's body or a center of mass of the patient's body part. The patient's body part may comprise the patient's head, the patient's arm(s), the patient's spine, the patient's hip(s), the patient's knee(s), the patient's foot or feet, the patient's joint(s), the patient's fingertip(s), the patient's nose, or a combination thereof. The patient's body part may comprise the patient's head, the patient's spine, the patient's spine base, the patient's mid-spine, the patient's neck, the patient's left shoulder, the patient's right shoulder, the patient's left elbow, the patient's right elbow, the patient's left wrist, the patient's right wrist, the patient's left hand, the patient's right hand, the patient's left hand tip, the patient's right hand tip, the patient's left thumb, the patient's right thumb, the patient's left hip, the patient's right hip, the patient's left knee, the patient's right knee, the patient's left ankle, the patient's right ankle, the patient's left foot, the patient's right foot, or a combination thereof.

In this disclosure, the spatial position information may comprise visual information representing the patient's body. The spatial position information may comprise visual information representing the patient's body, the patient's weight, the patient's height, the patient's body-mass-index (BMI), or a combination thereof. The system may be configured to generate spatial position information of at least two spatial positions, determine at least one kinematic parameter for each spatial position, compare these kinematic parameters with each other, and determine whether the patient will need unplanned medical care during a therapy and/or during a future period of time based on this comparison. The system may further be configured to generate spatial position information of a reference site unrelated to the patient; and determine whether the patient will need unplanned medical care based on the kinematic parameter determined by using the prescribed movement site relative to the reference site. The at least one kinematic parameter of the at least one spatial position may comprise velocity, acceleration, specific kinetic energy, specific potential energy, sagittal angle, angular velocity, or a combination thereof.

In this disclosure, the at least one kinematic parameter may comprise acceleration of the patient's non-pivoting knee, acceleration of the patient's non-pivoting hip, angular velocity of the patient's hip, angular velocity of the patient's non-pivoting leg, or a combination thereof. The at least one kinematic parameter may comprise chair-to-table acceleration of the patient's non-pivoting knee, chair-to-table acceleration of the patient's non-pivoting hip, chair-to-table angular velocity of the patient's hip, chair-to-table angular velocity of the patient's non-pivoting leg, or a combination thereof.

In this disclosure, the determination of the at least one kinematic parameter may comprise determining spatial position vectors for the at least one spatial position; and determining acceleration of the at least one spatial position based on the spatial position vectors using a mean-value theorem. The spatial position vectors may comprise three-dimensional time series generated for given positions of the at least one spatial position at a given time point during the prescribed movement; and the acceleration of the at least one spatial position is determined using the mean-value theorem based on the spatial position vectors of the spatial position of the center of mass.

In this disclosure, the determination of the kinematic parameter may comprise less bytes of data than the spatial position information conveyed by the at least one output signal.

In this disclosure, the at least one physical activity parameter may comprise at least one metabolic equivalent of task (MET). The determination of the at least one physical activity parameter is indicative of the physical activity of the patient.

In this disclosure, the determination of whether the patient will need unplanned medical care during therapy and/or the future period of time is based on the kinematic parameter; and/or the at least one physical activity of the patient. The system may further be configured to categorize the patient as either likely to need unplanned medical care or unlikely to need unplanned medical care during the therapy, wherein the categorization comprises determining Eastern Cooperative Oncology Group (ECOG) scores. The patient will need unplanned medical care during the therapy may comprise comparing the acceleration of the spatial position of the center of mass to an acceleration threshold, and determining the patient will need unplanned medical care during the therapy responsive to a breach of the acceleration threshold. The determining whether the patient will need unplanned medical care may comprise comparing a spine base acceleration time series to a corresponding baseline, determining a distance between the spine base acceleration time series and the corresponding baseline using Euclidean metric dynamic time warping (DTW), which assigns a distance of zero for completely identical series and larger distances for more dissimilar series, and determining the patient will need unplanned medical care during the therapy responsive to a breach of one or more DTW distance thresholds.

In this disclosure, the unplanned medical care may comprise a medical care unrelated to the therapy, an unscheduled medical care, a non-routine medical care, an emergency medical care, or a combination thereof.

In this disclosure, the system may further be configured to facilitate adjustment of the therapy based on the determination of whether the patient will need unplanned medical care during the therapy.

In this disclosure, the determination of whether the patient will need unplanned medical care during the therapy may be indicative of a future reaction of the patient to planned (e.g. targeted) therapeutic intervention. The determination of whether the patient will need unplanned medical care during the therapy may be indicative of a future reaction of the patient to planned (e.g. targeted) therapeutic intervention; and wherein the target therapeutic intervention comprises chemotherapy, radiation therapy, immune therapy, hormone therapy, or a combination thereof. The determination of whether the patient will need unplanned medical care during the therapy may be indicative of a future reaction of the patient to chemotherapy and/or radiation during the therapy. The determining whether the patient will need unplanned medical care during the therapy may comprise determining whether the patient will need unplanned medical care during a future period of time that corresponds to at least one therapy treatment received by the patient.

In this disclosure, the determining whether the patient will need unplanned medical care during the therapy may comprise determining a likelihood the patient will need unplanned medical care; and categorizing the patient into two or more groups based on the likelihood. The likelihood may comprise a numerical value on a continuous scale; and the likelihood may inversely be correlated to the acceleration of the spatial position of the center of mass.

This disclosure further relates to a quantitative health assessment method for quantitative determination of health-related performance or quality of life of a patient. The method may comprise using a quantitative health assessment system of any of the systems disclosed in this disclosure; and determining whether the patient will need unplanned medical care during a therapy and/or during a future period of time. The patient may be a clinical trial subject. The method may further comprise deciding whether to continue, stop, or modify the therapy. The method may further comprise deciding whether to stop or modify the therapy. The method may further comprise deciding whether to stop the therapy. The method may further comprise deciding whether to enroll the patient in a clinical trial. The method may further comprise deciding whether to terminate the subject's participation in a clinical trial.

In this disclosure, the therapy may be a therapy related to a clinical trial; and wherein the method further comprises deciding whether to stop or modify the clinical trial. The therapy may be a therapy related to a clinical trial; and wherein the method further comprises determining a total number of unplanned medical care occurred during the clinical trial; and using this total number in deciding whether the therapy provided a better/improved health-related quality of life to the patient as compared to another therapy.

In this disclosure, the future period of time is about two months.

In this disclosure, the reference site may comprise an exam table, a patient bed, a computer, or a combination thereof.

In this disclosure, the therapy may comprise a cancer therapy.

In this disclosure, the patient may be a clinical trial subject.

In this disclosure, the user may comprise a healthcare practitioner and/or the patient.

These and other objects, features, and characteristics of the system and/or method disclosed herein, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates an exemplary system configured to determine whether a cancer patient will need unplanned medical care during cancer therapy, in accordance with one or more embodiments.

FIG. 2 illustrates an exemplary wire-frame representation of a patient with anatomical sites and corresponding body parts labeled.

FIG. 3 illustrates a patient performing an exemplary prescribed movement associated with a chair to table exam.

FIG. 4 illustrates an exemplary wire frame representation of patient at four different time points during a prescribed movement similar to the prescribed movement shown in FIG. 3.

FIG. 5 illustrates an exemplary time series for the acceleration of the spine base of a cancer patient and a baseline dataset for the same cancer patient.

FIG. 6 illustrates an exemplary method for determining whether a cancer patient will need unplanned medical care during cancer therapy with a determination system.

FIG. 7A-B illustrates kinematic features that differentiate patients with zero unexpected hospitalizations from patients with one or more hospitalizations. A) ROC curves for features with the highest AUC. B) Boxplots for features with the highest t-test scores (UHV=0: gray, UHV=1: red). (vel: velocity; acc: acceleration; pe: potential energy; ke: kinetic energy; sa: sagittal angle; av-x, av-y, av-z: angular velocity about x,y, or z axes).

FIG. 8A-B illustrates top three kinematic features that differentiate patients with 15 hours or more of activity above LPA from patients with 15 hours or less of activity above LPA. A) ROC curves for features with the highest AUC. B) Boxplots for features with the highest t-test scores (HALPA=0: gray, HALPA=1: red). (vel: velocity; acc: acceleration; pe: potential energy; ke: kinetic energy; sa: sagittal angle; av-x, av-y, av-z: angular velocity about x,y, or z axes).

FIG. 9 illustrates distribution of t-test scores and significance values from two-sample t-tests for differences in mean values of kinematic features between patients with no unexpected hospitalizations (UHV=0) and patients with one or more unexpected hospitalizations (UHV=1).

FIG. 10 illustrates box plots of kinematic features that significantly differentiate between patients with no unexpected hospitalizations (UHV=0, gray) and patients with one or more unexpected hospitalizations (UHV=1, red). Kinematic features 1-20.

FIG. 11 illustrates box plots of kinematic features that significantly differentiate between patients with no unexpected hospitalizations (UHV=0, gray) and patients with one or more unexpected hospitalizations (UHV=1, red). Kinematic features 21-40.

FIG. 12 illustrates box plots of kinematic features that significantly differentiate between patients with no unexpected hospitalizations (UHV=0, gray) and patients with one or more unexpected hospitalizations (UHV=1, red). Kinematic features 41-55.

FIG. 13 illustrates distribution of t-test scores and significance values from two-sample t-tests for differences in mean values of kinematic features between patients with 15 hours or more of activity above LPA (HALPA=0) from patients with 15 hours or less of activity above LPA (HALPA=1).

FIG. 14 illustrates box plots of kinematic features that significantly differentiate between patients with 15 hours or more of activity above LPA (HALPA=0, gray) from patients with 15 hours or less of activity above LPA (HALPA=1, red). Kinematic features 1-20.

FIG. 15 illustrates box plots of kinematic features that significantly differentiate between patients with 15 hours or more of activity above LPA (HALPA=0, gray) from patients with 15 hours or less of activity above LPA (HALPA=1, red). Kinematic features 21-28.

### DETAILED DESCRIPTION

The term “a”, “an” or “the” is intended to mean “one or more”, e.g., a chair refers to one or more chairs unless otherwise made clear from the context of the text.

The term “comprise,” and variations thereof such as “comprises” and “comprising,” when preceding the recitation of a step or an element, are intended to mean that the addition of further steps or elements is optional and not excluded.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Any methods and reagents similar or equivalent to those described herein can be used in the practice of the disclosed methods and compositions.

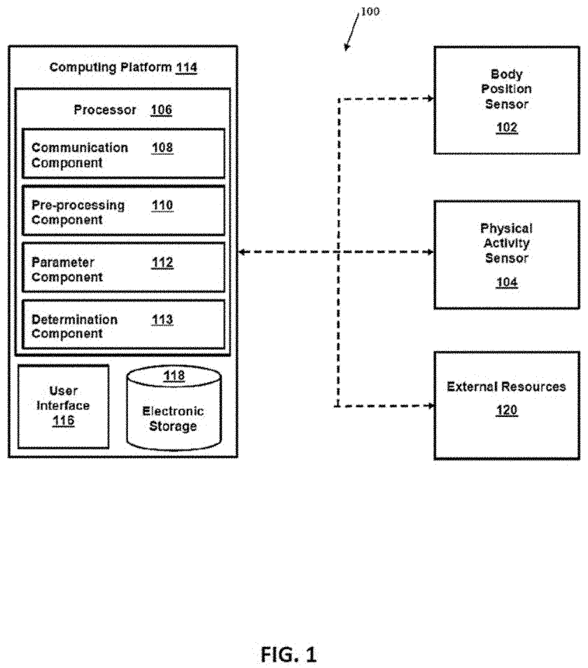

FIG. 1 illustrates an exemplary system 100 configured to determine whether a cancer patient will need unplanned medical care during cancer therapy. Poor patient outcomes, patient satisfaction, quality of life, and economic cost are associated with unplanned medical care for patients actively receiving cancer therapy (e.g., chemotherapy). Predicting a patient's needs during cancer therapy, and providing specific solutions to those needs may improve patient outcomes and the patient's experience during treatment.

Observing the way a patient moves provides a clinician with valuable information about frailty. This is important for patients undergoing difficult treatments such as chemotherapy. A comprehensive geriatric (e.g., frailty) assessment can predict complications and side effects from cancer treatment. However, clinicians' assessments are often qualitative, subjective, and lack agreement among clinicians. Available tools and metrics such as the Eastern Cooperative Oncology Group (ECOG) performance status, body mass index (BMI) measurements, Mini Mental State Exam (MMSE) results, and the Charlson Comorbidity Index (CCI), are often part of a comprehensive geriatric assessment, but few clinicians perform a complete assessment because such assessments are time consuming.

Laboratory based invasive methods have been developed to biomechanically quantify elements of human performance. Many of these methods comprise conducting gait analysis using an accelerometer, a gyroscope, and other types of wearable sensors and motion capture systems to detect and differentiate conditions in patients with osteoarthritis, neuromuscular disorders, and cerebral palsy. However, these methods are associated with high cost, lengthy time required to perform tests, and general difficulty in interpreting results.

Although these tools and metrics are known, and continue to be used because of their practicality, standardization of patient stratification, and speed of assessment; inter- and intra-observer variability, gender discrepancies, sources of subjectivity in physician assigned performance assessments, and a lack of standard conversions between different evaluation scales continue to exist. As such, there is a need for a system and method for more objective classification of a patient's physical function that may be used to guide decisions about oncological therapy in an effort to reduce or avoid a need for unplanned medical care.

Advantageously, the system 100 is a non-invasive motion-capture based performance assessment system which can (i) determine kinematic parameters that characterize a cancer patient's biomechanical performance and/or physical activity parameters that characterize a level of physical activity of the cancer patient, and (ii) determine whether a cancer patient will need unplanned medical care during cancer therapy based on the kinematic and/or physical activity parameters.

In this disclosure, the system 100 comprises one or more of a body position sensor 102; a physical activity sensor 104; computing platform 114 comprising a processor 106, a user interface 116 and electronic storage 118; external resources 120; and/or other components.

Body position sensor 102 may be configured to generate one or more output signals conveying spatial position information and/or other information. The spatial position information and/or other information may be a time series of information that conveys spatial position information about the body and/or body parts of a cancer patient over time. In this disclosure, the spatial position information may comprise visual information representing the body and/or individual body parts of the cancer patient, and/or other information. The visual information representing the cancer patient may include one or more of still images, video images, and/or other information. For example, body position sensor 102 may be configured such that the spatial position information includes body position signals conveying information associated with the position of one or more body parts of the cancer patient relative to each other and/or other reference locations. In this disclosure, the visual information may be and/or include a wire-frame representation of the cancer patient and/or other visual information. According to some embodiments, body position sensor 102 may include an infrared stereoscopic sensor configured to facilitate determination of user body positions, such as for example the Kinect™ available from Microsoft™ of Redmond, Wash., and/or other sensors.

Body position sensor 102 may be configured such that the spatial information comprises information associated with one or more body positions and/or other physical characteristics of the cancer patient. The spatial position information in the output signals may be generated responsive to a prescribed movement performed by the cancer patient and/or at other times. A given body position may describe, for example, a spatial position, orientation, posture, and/or other positions of the cancer patient and/or of one or more body parts of the cancer patient. A given physical characteristic may include, for example, a size, a length, a weight, a shape, and/or other characteristics of the cancer patient, and/or of one or more body parts of the cancer patient. The output signals conveying the spatial position information may include measurement information related to the physical size, shape, weight, and/or other physical characteristics of the cancer patient, movement of the body and/or one or more body parts of the cancer patient, and/or other information. The one or more body parts of the cancer patient may include a portion of the first user's body (e.g., one or more of a head, neck, torso, foot, hand, head, arm, leg, and/or other body parts).

The spatial position information may be related to spatial positions of one or more anatomical sites on the cancer patient. The one or more anatomical sites may be and/or correspond to the body parts described above, for example. The one or more anatomical sites may comprise an anatomical site (e.g., a body part) that is indicative of a patient's mobility, corresponds to a center of mass of the cancer patient, and/or include other anatomical sites. In this disclosure, locations that are indicative of a patient's mobility and/or correspond to the center of mass may be a location at a base of a spine of the cancer patient, a location near a hip or hips, a location near a knee, and/or other locations.

Technological advances in low cost spatial cameras, such as Microsoft Kinect, have the potential to objectively define and categorize patients with varying levels of mobility at home or in the clinic. Similarly, low cost activity trackers containing accelerometers, such as Microsoft Band, can capture daily movement in the clinic and at home, assessing dynamic changes related to exertion or to physical challenges such as the chemotherapy cycle. These consumer technologies have the capacity to bring objectivity to the assessment of mobility and performance status of patients on chemotherapy.

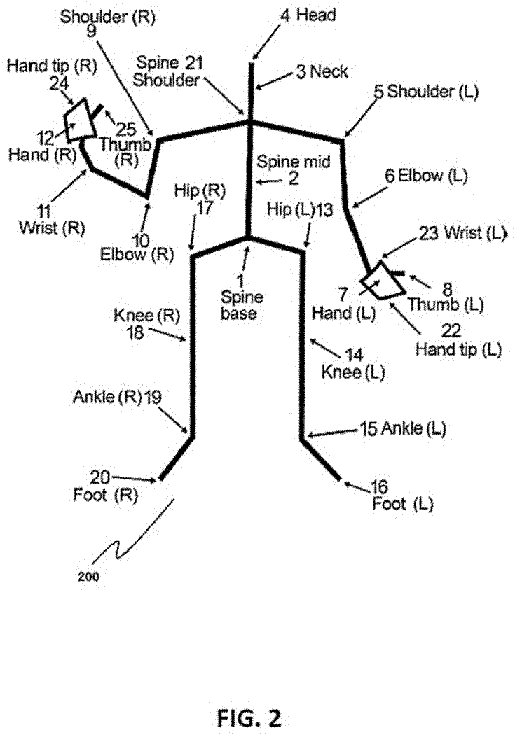

By way of a non-limiting example, FIG. 2 illustrates a wire-frame representation 200 of a patient with anatomical sites 1-20 and corresponding body parts labeled. FIG. 2 illustrates spatial positions of one or more anatomical sites 1-20 on the cancer patient. As described above, the spatial position information in the output signals from body position sensor 102 may comprise visual information representing the body and/or individual body parts of the cancer patient. Wire-frame representation 200 may be and/or be included in such visual information. As shown in FIG. 2, anatomical site 1 corresponds to the base of the patient's spine, anatomical site 2 corresponds to the patient's mid-spine, and so on. Wire frame representation 200 may correspond to a given body position and may describe, for example, a spatial position, orientation, posture, and/or other positions of the cancer patient and/or of one or more body parts of the cancer patient. Wire-frame representation 200 may provide information related to the physical size, shape, weight, and/or other physical characteristics of the cancer patient (e.g., height may represented as a distance from anatomical sites 16 or 20 corresponding to the left or right foot to the anatomical site 4 corresponding to the head), movement of the body and/or one or more body parts of the cancer patient (e.g., movement of anatomical site 1 corresponding to the spine base), relative positions of one or more body parts of the cancer patient, and/or other information. As described above, anatomical site 1, which corresponds to the spine base of the patient, corresponds to a center of mass of the cancer patient. Other anatomical sites indicative of mobility and/or a center of mass of a cancer patient are also contemplated—e.g., a knee, a hip, etc.

The spatial position information (e.g., from body position sensor 102 shown in FIG. 1) may be related to spatial positions of the one or more anatomical sites on the cancer patient while the cancer patient performs the prescribed movement and/or at other times. The prescribed movement may comprise movement associated with a chair to table (CTT) exam, a get up and walk (GUP) exam, and/or other movement, for example.

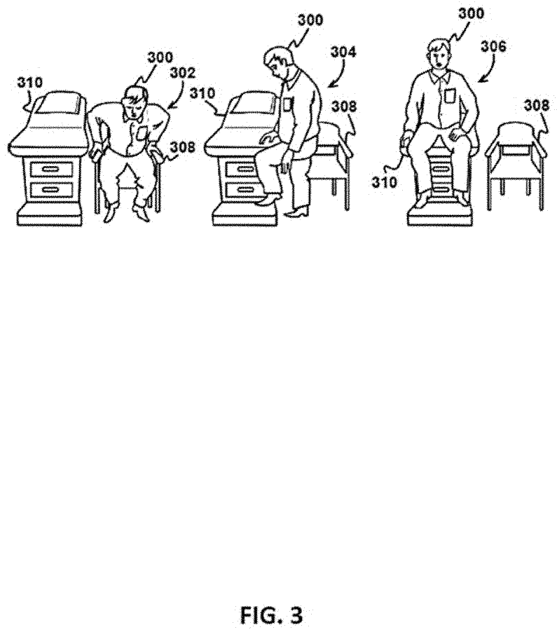

By way of a non-limiting example, FIG. 3 illustrates a patient 300 performing a prescribed movement 302, 304, 306 associated with a chair to table exam. Patient 300 starts in a sitting position in a chair 308 and begins to stand 302. Patient 300 then moves toward, and steps up onto 304 an exam table 310. Patent 300 finishes the prescribed movement by sitting 306 on exam table 310.

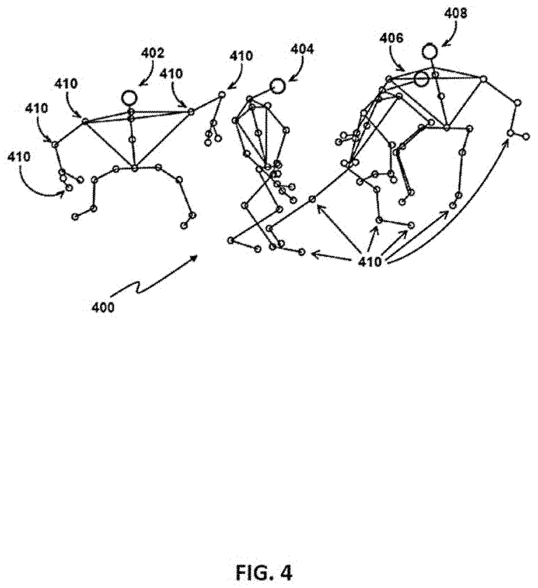

FIG. 4 illustrates a wire frame representation 400 of patient (e.g., 300 shown in FIG. 3) at four different time points 402, 404, 406, 408 during a prescribed movement similar to prescribed movement 302, 304, 306 shown in FIG. 3. In FIG. 4, wire frame representation 400 starts in a sitting position (e.g., in a chair that is not shown in FIG. 4) and begins to stand 402, then moves toward 404 and steps up 406 onto an exam table (not shown in FIG. 4), and finishes the prescribed movement by sitting 408 on the exam table. In FIG. 4, wire frame representation 400 is shown moving toward 404 and stepping onto 402 an exam table (not shown in FIG. 4) from the opposite direction shown in FIG. 3. Wire-frame representation 400 illustrates anatomical sites 1-20 illustrated in FIG. 2 as dots 410 at each time point 402, 404, 406, and 408 of the prescribed movement shown in FIG. 4. Wire-frame representation 400 may be and/or be included in the spatial information in the output signals from body position sensor 102 (FIG. 1) described above. Processor 106 (shown in FIG. 1 and described below) may be configured to use wire frame representation 400, for example, and/or other information to determine one or more parameters related to the movement (e.g., a velocity, an acceleration, etc.) of one or more anatomical sites 410. In this disclosure, processor 106 may determine an acceleration of anatomical site 1 (as described herein), which corresponds to the spine base of a cancer patient, and corresponds to a center of mass of the cancer patient. In this disclosure, processor 106 may determine a velocity and/or an acceleration of a knee, a hip, a spine base, and/or other anatomical sites of the cancer patient

Returning to FIG. 1, physical activity sensor 104 may be configured to generate one or more output signals that convey physical activity information and/or other information related to the cancer patient. The physical activity information may be related to physical activity performed by the cancer patient and/or other information. Physical activity performed by the cancer patient may include any movement, motion, and/or other activity performed by the cancer patient. Physical activity may include exercise, normal daily activities, and/or other physical activities. Exercise may include, for example, walking, running, biking, stretching, and/or other exercises. Normal daily activities may include movement through the house, household chores, commuting, working at a computer, shopping, making a meal, and/or other normal daily activities. In this disclosure, physical activity may include maintaining a given posture for a period of time. For example, physical activity may include sitting, standing, lying down, and/or maintaining other postures for a period of time. In this disclosure, physical activity sensor 104 may comprise a wrist worn motion sensor and/or other sensors, for example. In this disclosure, physical activity sensor 104 is and/or includes the Microsoft Band™ available from Microsoft™ of Redmond, Wash., and/or other similar sensors.

In this disclosure, as described above, body position sensor 102 and/or physical activity sensor 104 may be stand-alone devices, separate from one or more other components of system 100, and communicate with one or more other components of system 100 (e.g., computing platform 114) as a peripheral device. In this disclosure, body position sensor 102 and/or physical activity sensor 104 may be integrated with computing platform 114 as a single device (e.g., as a camera that is part of computing platform 114, as an activity tracking sensor built into computing platform 114, etc.). In this disclosure, body position sensor 102, physical activity sensor 104, and/or computing platform 114 may be associated with the cancer patient and/or may be carried by the cancer patient. For example, body position sensor 102 and/or physical activity sensor 104 may be included in a Smartphone associated with the cancer patient. As such, information related to physical activity of the cancer patient may be obtained throughout the day as the cancer patient goes about his daily business and/or participates in specific activities.

Although body position sensor 102 and physical activity sensor 104 are depicted in FIG. 1 as individual elements, this is not intended to be limiting, as other embodiments that include multiple body position sensors 102 and/or physical activity sensors 104 are contemplated and within the scope of the disclosure. For example, In this disclosure, a given computing platform 114 may have one or more integrated body position sensors 102 and/or physical activity sensors 104, and/or be in communication with one or more additional body position sensors 102 and/or physical activity sensors 104 as separate peripheral devices.

Computing platform 114 may include one or more processors 106, a user interface 116, electronic storage 118, and/or other components. Processor 106 may be configured to execute computer program components. The computer program components may be configured to enable an expert or user associated with a given computing platform 114 to interface with system 100 and/or external resources 120, and/or provide other functionality attributed herein to computing platform 114. By way of non-limiting example, computing platform 114 may include one or more of a desktop computer, a laptop computer, a handheld computer, a tablet computing platform, a Smartphone, a gaming console, and/or other computing platforms.

Processor 106 is configured to provide information-processing capabilities in computing platform 114 (and/or system 100 as a whole). As such, processor 106 may comprise one or more of a digital processor, an analog processor, a digital circuit designed to process information, an analog circuit designed to process information, a state machine, and/or other mechanisms for electronically processing information. Although processor 106 is shown in FIG. 1 as a single entity, this is for illustrative purposes only. In this disclosure, processor 106 may comprise a plurality of processing units. These processing units may be physically located within the same device (e.g., computing platform 114), or processor 106 may represent processing functionality of a plurality of devices operating in coordination (e.g., a processor included in computing platform 114, a processor included in body position sensor 102, a processor included in physical activity sensor 104, etc.). In this disclosure, processor 106 may be and/or be included in a computing device such as computing platform 114 (e.g., as described herein). Processor 106 may run one or more electronic applications having graphical user interfaces configured to facilitate user interaction with system 100.

As shown in FIG. 1, processor 106 is configured to execute one or more computer program components. The computer program components may comprise software programs and/or algorithms coded and/or otherwise embedded in processor 106, for example. The computer program components may include one or more of a communication component 108, a pre-processing component 110, a parameter component 112, a determination component 113, and/or other modules. Processor 106 may be configured to execute components 108, 110, 112, and/or 113 by software; hardware; firmware; some combination of software, hardware, and/or firmware; and/or other mechanisms for configuring processing capabilities on processor 106.

It should be appreciated that although components 108, 110, 112, and 113 are illustrated in FIG. 1 as being co-located in processor 106, one or more of the components 108, 110, 112, or 113 may be located remotely from the other components. The description of the functionality provided by the different components 108, 110, 112, and/or 113 described below is for illustrative purposes, and is not intended to be limiting, as any of the components 108, 110, 112, and/or 113 may provide more or less functionality than is described, which is not to imply that other descriptions are limiting. For example, one or more of the components 108, 110, 112, and/or 113 may be eliminated, and some or all of its functionality may be provided by others of the components 108, 110, 112, and/or 113. As another example, processor 106 may include one or more additional components that may perform some or all of the functionality attributed below to one of the components 108, 110, 112, and/or 113.

Communication component 108 may be configured to facilitate bi-directional communication between computing platform 114 and one or more other components of system 100. In this disclosure, the bi-directional communication may facilitate control over one or more of the other components of system 100, facilitate the transfer of information between components of system 100, and/or facilitate other operations. For example, communication component 108 may facilitate control over body position sensor 102 and/or physical activity sensor 104 by a user (e.g., the cancer patient, a doctor, a nurse, a caregiver, etc.). The control may be based on entries and/or selections made by the user via user interface 116, for example, and/or based on other information. As another example, communication component 108 may facilitate uploading and/or downloading data to or from body position sensor 102, physical activity sensor 104, external resources 120, and/or other components of system 100.

Continuing with this example, communication component 108 may be configured to receive the spatial information and/or the physical activity information in the output signals from body position sensor 102 and/or physical activity sensor 104. The output signals may be received directly and/or indirectly from body position sensor 102 and/or physical activity sensor 104. For example, body position sensor 102 may be built into computing platform 114, and the output signals from body position sensor 102 may be transmitted directly to communication component 108. As another example, physical activity sensor 104 may be a separate wrist worn device. The output signals from the wrist worn device may be wirelessly transmitted to communication component 108.

In this disclosure, communication component 108 may be configured to cause display (e.g., on user interface 116) of the spatial information, the physical activity information, a determination, and/or other information. In this disclosure, communication component 108 may be configured to cause display (e.g., on user interface 116) of a graphical control interface to facilitate user control of body position sensor 102, physical activity sensor 104, and/or other components of system 100.

Pre-processing component 110 is configured to pre-process the spatial information, the physical activity information, and/or other information received by communication component 108. In this disclosure, pre-processing comprises filtering, converting, normalizing, adjusting, and/or other pre-processing operations performed on the spatial information, the physical activity information, and/or other information in the output signals from body position sensor 102, physical activity sensor 104, and/or other components of system 100. In this disclosure, pre-processing component 110 may be configured to automatically segment (and/or facilitate manually segmenting) the spatial information to trim irrelevant data at the beginning and end of a prescribed movement while a patient is stationary. Preprocessing component 110 may be configured to pre-process the spatial information to compensate for irregularities in the spatial information caused by the positioning of body position sensor 102 relative to a given cancer patient, features of an environment or location where the prescribed movement occurs, and/or other factors. In this disclosure, pre-processing component 110 may be configured such that pre-processing includes coordinate transformation for three-dimensional data coordinates included in the spatial information. For example, the spatial information received by communication component 108 may be distorted such that a level plane such as a clinic floor appears sloped in the spatial information, for example. In this example, the angle of distortion, θ, may range between about 5° and about 20°. Pre-processing component 110 may be configured to resolve this distortion by performing an automated element rotation about an x-axis of the spatial information. As other examples, in this disclosure, pre-processing may include filters to remove other background humans from the images prior to analysis during the CTT exam; and, for a wrist worn sensor (e.g., as described herein), pre-processing may include adjustments for weight, gender, race, time, diet, and location prior to calculation of metabolic equivalents.

Parameter component 112 may be configured to determine one or more kinematic parameters, physical activity parameters, and/or other parameters. Parameter component 112 may be configured to determine the one or more kinematic and/or physical activity parameters based on the information in the output signals from body position sensor 102 and/or physical activity sensor 104, the pre-processing performed by pre-processing component 110, and/or other information. In this disclosure, the one or more determined kinematic and/or physical activity parameters may be features extracted from the spatial position or physical activity information, and/or other parameters. In this disclosure, the determined kinematic and/or physical activity parameters may comprise less bytes of data than the spatial position information and/or the physical activity information conveyed by the one or more output signals.

In this disclosure, parameter component 112 may be configured to determine one or more kinematic parameters indicative of the movement of the cancer patient during the prescribed movement based on the spatial position information and/or other information. The one or more kinematic parameters may comprise one or more positions of a given anatomical site (e.g., 1-20 shown in FIG. 2) over time, velocities of anatomical sites during the prescribed movement, accelerations (e.g., in any direction) of anatomical sites during the prescribed movement, kinetic energies, potential energies, sagittal angles, and/or other kinematic parameters. For example, parameter component 112 may be configured to determine an acceleration (in any direction) of an anatomical site that corresponds to the center of mass of the cancer patient and/or other parameters. In this disclosure, parameter component 112 may be configured to determine relative accelerations (and/or any other motion related parameter) of one or more anatomical sites. For example, parameter component 112 may be configured to determine a first acceleration of a first anatomical site relative to one or more second accelerations of one or more second anatomical sites. In this disclosure, parameter component 112 may be configured to determine acceleration of an anatomical site relative to a reference site (e.g., an exam table, a patient bed, a computer, and/or other reference sites).

In this disclosure, determining the one or more kinematic parameters indicative of the movement of the cancer patient during the prescribed movement based on the spatial position information comprises determining anatomical site position vectors for the one or more anatomical sites. The anatomical site position vectors may comprise three-dimensional time series generated for given positions of the one or more anatomical sites at time points (e.g., 402, 404, 406, 408 shown in FIG. 4) during the prescribed movement. This may also include determining accelerations for the one or more anatomical sites based on the anatomical site position vectors using a mean-value theorem. For example, parameter component 112 may be configured such that the acceleration of the spine base (e.g., anatomical site 1 shown in FIG. 2 that corresponds to the center of mass of the cancer patient) is determined using the mean-value theorem based on the anatomical site position vectors for the spine base. (Other anatomical sites indicative of mobility and/or a center of mass of a cancer patient are also contemplated—e.g., a knee, a hip, etc.)

By way of a non-limiting example, a position vector

$$\vec{{r}_{i}} ( t ) = 〈 {x}_{i} ( t ) , {y}_{i} ( t ) , {z}_{i} ( t ) 〉$$

for an anatomical site i may be used to calculate the anatomical site's velocity magnitude,

$${v}_{i} ( t ) = ‖ \vec{{\dot{r}}_{i}} ( t ) ‖$$

and acceleration magnitude,

$${a}_{i} ( t ) = ‖ \vec{{\dot{r}}_{i}} ( t ) ‖$$

using the mean-value theorem. In the absence of distribution of mass information, specific kinetic energy,

$$k {e}_{i} ( t ) = \frac{1}{2} {v}_{i}^{2} ( t )$$

and specific potential energy

$$p {e}_{i} ( t ) = g Δ {z}_{i} = g ( {z}_{i} ( t ) - {z}_{i} ( t = 1 ) )$$

quantities may be used to describe the energy signature of each anatomical site. Parameter component 112 may be configured such that the sagittal angle, θₛ(t), is defined as the angle formed between the vector originating at the spine base and pointing in the direction of motion, and the vector connecting the anatomical sites for the spine base (e.g., 1 in FIG. 2) and the neck (e.g., 3 in FIG. 2) at each time point t (e.g., 402, 404, 406, 408 shown in FIG. 4).

In this disclosure, parameter component 112 may be configured to determine one or more physical activity parameters indicative of the physical activity of the cancer patient based on the physical activity information and/or other information. In this disclosure, the one or more physical activity parameters may comprise an amount of time a cancer patient engages in physical activity, a level (e.g., low or high, above or below a predetermined threshold level, etc.) of the physical activity, an amount of energy expended during the physical activity, an amount of calories burned during the physical activity, metabolic equivalence (METs) associated with the physical activity, and/or other parameters. In this disclosure, parameter component 112 may be configured to aggregate (e.g., sum, average, etc.), normalize, and/or perform other operations for the one or more physical activity parameters for a given evaluation period (e.g., per hour, per day, per week, for the time between doctor visits, etc.). In this disclosure, parameter component 112 may be configured to aggregate a given physical activity parameter for the evaluation period only for instances of physical activity that breach a predetermined threshold level during the evaluation period.

For example, in this disclosure, parameter component 112 may be configured to determine total (e.g., a summation of) METs associated with physical activity performed by the cancer patient during the evaluation period. In this disclosure, a total number of METs may be an indication of any and all physical activity by a cancer patient during an evaluation period. METs provide an indication of an amount of energy consumed while sitting at rest relative to an amount of energy consumed while performing a physical activity. In this disclosure, METs may be calculated based on a determination of mechanical work completed. One MET, for example, is equal to 1.1622 watts/kg, where a watt of work is equal to the energy required to move an object at constant velocity of one meter/second against a force of one Newton. Acceleration against force may be determined by integration of a directional force vector from a three-axis accelerometer sensor (e.g., as described herein) and correcting for the weight of the wearer, for example.

In this disclosure, parameter component 112 may be configured such that only METs associated with high levels of physical activity (e.g., physical activity that breaches a predetermined threshold level) may be included in the total. In this disclosure, parameter component 112 may be configured to determine total daily, weekly, or monthly active hours above a threshold of, for example, 1.5 METs (light), 3METs (moderate), or 6 METs (vigorous) physical activity. In this disclosure, parameter component 112 may determine a fraction of daytime hours spent in non-sedentary activity. Total distance travelled and steps taken may be alternative measures of activity, for example.

The physical activity parameters determined by parameter component 112, aggregation operations, threshold levels, and/or other characteristics of parameter component 112 may be determined at manufacture of system 100, determined and/or adjusted by a user via user interface 116, and/or determined in other ways.

Determination component 113 may be configured to determine whether a cancer patient will need unplanned medical care. In this disclosure, the determination of whether the cancer patient will need unplanned medical care during cancer therapy is indicative of a future reaction of the cancer patient to chemotherapy and/or radiation during cancer therapy. In this disclosure, the determining may be based on the acceleration (in any direction) of the anatomical site that corresponds to the center of mass of the cancer patient (e.g., the spine base) and/or other information. In this disclosure, determination component 113 may be configured to determine whether the cancer patient will need unplanned medical care during cancer therapy based on relative accelerations (and/or any other motion parameters) of anatomical sites. For example, determination component 113 may be configured to determine whether the cancer patient will need unplanned medical care based on a comparison of a first acceleration of a first anatomical site to one or more second accelerations of one or more second anatomical sites. In this disclosure, determination component 113 may be configured to determine whether a cancer patient will need unplanned medical care based on acceleration of an anatomical site relative to a reference site (e.g., an exam table, a patient bed, a computer, and/or other reference sites).

In this disclosure, the determining may be based on the metabolic equivalence determined for the cancer patient, and/or other information.

In this disclosure, determining whether the cancer patient will need unplanned medical care during cancer therapy may comprise determining whether the cancer patient will need unplanned medical care during a future period of time that corresponds to one or more cancer therapy treatments received by the cancer patient. In this disclosure, the future period of time is about two months and/or other periods of time. This example is not intended to be limiting.

In this disclosure, determination component 113 may be configured such that determining whether the cancer patient will need unplanned medical care comprises comparing the acceleration of the center of mass of the cancer patient to an acceleration threshold, comparing the METs for the cancer patient to a METs threshold, and/or comparing other parameters to other thresholds, and determining the cancer patient will need unplanned medical care during cancer therapy responsive to a breach of one or more of the thresholds. By way of a non-limiting example, in this disclosure, the spine base acceleration threshold may be about one meter per second squared (1 m/s²), and the METs threshold may be about zero waking hours above 1.5METs (these are merely examples). Determination component 113 may be configured such that if the acceleration of the spine base is in breach of (e.g., below in this example) the spine base acceleration threshold, and/or if the METs are in breach of (e.g., below in this example) the METs threshold, the cancer patient is determined to need unplanned medical care. These examples are not intended to be limiting. The thresholds may be any thresholds on any parameters that are indicative of whether the cancer patient will need unplanned medical care during cancer therapy. In this disclosure, the thresholds may be determined at manufacture of system 100, determined and/or adjusted based on entries and/or selections made by a user via user interface 116, learned by determination component 113 (e.g., as described below), and/or determined in other ways.

In this disclosure, determination component 113 may be configured such that determining whether the cancer patient will need unplanned medical care comprises comparing a spine base acceleration (and/or other parameter) time series (e.g., determined as described above) and/or a physical activity (e.g., as indicated by METs) over time dataset to a corresponding baseline and/or reference dataset. In this disclosure, determination component 113 may be configured to determine a distance between the spine base acceleration time series and/or the physical activity over time dataset and the corresponding baseline and/or reference dataset. For example, the time series for a given feature (e.g., the acceleration of the spine base) may be compared to a baseline and/or reference dataset using Euclidean metric dynamic time warping (DTW), which assigns a distance of zero for completely identical series and larger distances for more dissimilar series.

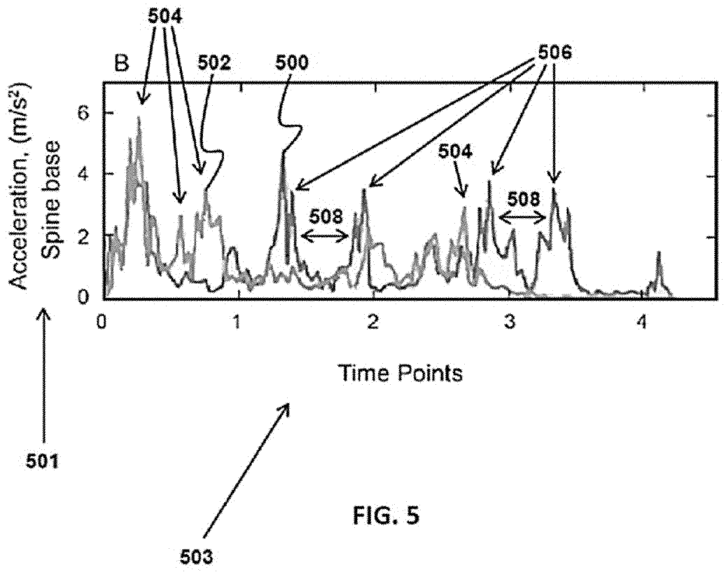

By way of a non-limiting example, FIG. 5 illustrates a time 503 series (e.g., at time points 1, 2, 3, and 4 shown in FIG. 5) 500 for the acceleration 501 of the spine base of a cancer patient and a baseline dataset 502 for the same cancer patient. Determination component 113 may be configured to use DTW to determine a distance between series 500 and 502. Series 500 and series 502 are not the same. They have peaks 504, 506 in different places relative to time points 1-4 and the distances 508 between peaks are not the same, for example. Since series 500 and 502 are not the same, as shown in FIG. 5, DTW would determine a non-zero distance value.

Returning to FIG. 1, determination component 113 may be configured to determine the cancer patient will need unplanned medical care during cancer therapy responsive to a breach of one or more of (DTW) distance thresholds. In this disclosure, the baseline and/or reference datasets, the distance thresholds, and/or other information may be determined at manufacture of system 100, determined and/or adjusted based on entries and/or selections made by a user via user interface 116, learned by determination component 113 (e.g., as described below), and/or determined in other ways.

In this disclosure, determination component 113 is configured to categorize the cancer patient as either likely to likely to need unplanned medical care or unlikely to need unplanned medical care during cancer therapy. In this disclosure, determination component 113 is configured to determine a likelihood (e.g., a numerical value on a continuous scale, a high-medium-low indication, a color representation of the likelihood, etc.) the cancer patient will need unplanned medical care, and categorize the cancer patient into two or more groups based on the likelihood. Determination component 113 may be configured such that the likelihood is inversely correlated to the acceleration of the spine base, the METs, and/or other parameters. For example, higher acceleration of a cancer patient's spine base indicates lower likelihood the cancer patient will need unplanned medical care. Similarly, the higher the number of METs for the cancer patient, the lower the likelihood the cancer patient will need unplanned medical care. In this disclosure, the categorization boundaries, the likelihood determination method, and/or other information may be determined at manufacture of system 100, determined and/or adjusted based on entries and/or selections made by a user via user interface 116, learned by determination component 113 (e.g., as described below), and/or determined in other ways.

In this disclosure, determination component 113 may be configured such that determining whether the cancer patient will need unplanned medical care and/or categorizing the cancer patient as either likely or unlikely to need unplanned medical care may include predicting ECOG scores. In this disclosure, the ECOG scores may be predicted based on the acceleration of the spine base of the cancer patient, the METs associated with the cancer patient, and/or other information, and the determination of whether or not the cancer patient will need unplanned medical care may be based on the ECOG scores.

In this disclosure, determination component 113 may be and/or include a trained prediction model. The trained prediction model may be an empirical model and/or other trained prediction models. The trained prediction model may perform some or all of the operations of determination component 113 described herein. The trained prediction model may predict outputs (e.g., whether or not the cancer patient will need unplanned medical care, ECOG scores, etc.) based on correlations between various inputs (e.g., the spatial information, the physical activity information, etc.).

As an example, the trained prediction model may be a machine learning model. In this disclosure, the machine learning model may be and/or include mathematical equations, algorithms, plots, charts, networks (e.g., neural networks), and/or other tools and machine learning model components. For example, the machine learning model may be and/or include one or more neural networks having an input layer, an output layer, and one or more intermediate or hidden layers. In this disclosure, the one or more neural networks may be and/or include deep neural networks (e.g., neural networks that have one or more intermediate or hidden layers between the input and output layers).

As an example, the one or more neural networks may be based on a large collection of neural units (or artificial neurons). The one or more neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In this disclosure, each individual neural unit may have a summation function that combines the values of all its inputs together. In this disclosure, each connection (or the neural unit itself) may have a threshold function such that a signal must surpass the threshold before it is allowed to propagate to other neural units. These neural network systems may be self-learning and trained, rather than explicitly programmed, and can perform significantly better in certain areas of problem solving, as compared to traditional computer programs. In this disclosure, the one or more neural networks may include multiple layers (e.g., where a signal path traverses from front layers to back layers). In this disclosure, back propagation techniques may be utilized by the neural networks, where forward stimulation is used to reset weights on the “front” neural units. In this disclosure, stimulation and inhibition for the one or more neural networks may be more free flowing, with connections interacting in a more chaotic and complex fashion. In this disclosure, the intermediate layers of the one or more neural networks include one or more convolutional layers, one or more recurrent layers, and/or other layers.

The machine learning model may be trained (i.e., whose parameters are determined) using a set of training data. The training data may include a set of training samples. The training samples may include spatial information and/or physical activity information, for example, for prior cancer patients, and an indication of whether the prior cancer patients needed unplanned medical care. Each training sample may be a pair comprising an input object (typically a vector, which may be called a feature vector, which may be representative of the spatial and/or physical activity information) and a desired output value (also called the supervisory signal)—for example indicating whether unplanned medical care was needed. A training algorithm analyzes the training data and adjusts the behavior of the machine learning model by adjusting the parameters of the machine learning model based on the training data. For example, given a set of N training samples of the form {(x₁, y₁), (x₂, y₂), . . . , (x_{N}, y_{N})} such that xᵢ is the feature vector of the i-th example and yᵢ is its supervisory signal, a training algorithm seeks a machine learning model g: X→Y, where X is the input space and Y is the output space. A feature vector is an n-dimensional vector of numerical features that represent some object (e.g., the spatial information and/or the physical activity information for a cancer patient as described above). The vector space associated with these vectors is often called the feature space. During training, the machine learning model may learn various parameters such as the spine base acceleration threshold, the METs threshold, the time series distance determination threshold, the categorization boundaries and/or other thresholds as described above. After training, the machine learning model may be used for making predictions using new samples. For example, the trained machine learning model may be configured to predict ECOG scores, whether or not a cancer patient will need unplanned medical care, and/or other information based on corresponding input spatial information and/or physical activity information for the cancer patient.

In this disclosure, determination component 113 may be configured to facilitate adjustment of the cancer therapy and/or other therapies. The adjustment may be based on the determination of whether the patient will need unplanned medical care and/or other information. In this disclosure, facilitating may comprise determining and displaying recommended changes, determining one or more additional parameters from the information in the output signals from the one or more sensors, and/or other operations. For example, based on the determination of whether the patient will need unplanned medical care, in treating a patient with a PD-L1 high expressing lung cancer, an oncologist may choose to treat a patient with a high risk with checkpoint inhibitor therapy alone, rather than a combination of chemotherapy with checkpoint inhibitor therapy. Similarly, a patient with an oral cavity squamous cell carcinoma undergoing combined chemo-radiation may be treated with a lower intensity weekly low-dose cisplatin regimen rather than a higher intensity regimen of high dose cisplatin given at 3 week intervals. Alternatively, physicians may decide to dose reduce chemotherapy to 80% (for example) of the usual standard dose prior to administration of the 1st cycle in anticipation of poor tolerability.

Body position sensor 102, physical activity sensor 104, and processor 106 may be configured to generate, determine, communicate, analyze, present, and/or perform any other operations related to the determinations, the spatial information, the physical activity information and/or any other information in real-time, near real-time, and/or at a later time. For example, the spatial information and/or physical activity information may be stored (e.g., in electronic storage 118) for later analysis (e.g., determination of a prediction). In this disclosure, the stored information may be compared to other previously determined information (e.g., threshold values, etc.), and/or other information.

As shown in FIG. 1, user interface 116 may be configured to provide an interface between computing platform 114 and a user (e.g., a doctor, a nurse, a physical therapy technician, the cancer patient, etc.) through which the user may provide information to and receive information from system 100. This enables data, cues, results, and/or instructions and any other communicable items, collectively referred to as “information,” to be communicated between the user and system 100. Examples of interface devices suitable for inclusion in user interface 116 include a touch screen, a keypad, buttons, switches, a keyboard, knobs, levers, a display, speakers, a microphone, an indicator light, an audible alarm, a printer, and/or other interface devices. In this disclosure, user interface 116 includes a plurality of separate interfaces. In this disclosure, user interface 116 includes at least one interface that is provided integrally with computing platform 114.

It is to be understood that other communication techniques, either hard-wired or wireless, are also contemplated by the present disclosure as user interface 116. For example, the present disclosure contemplates that user interface 116 may be integrated with a removable storage interface provided by computing platform 114. In this example, information may be loaded into computing platform 114 from removable storage (e.g., a smart card, a flash drive, a removable disk) that enables the user to customize the implementation of computing platform 114. Other exemplary input devices and techniques adapted for use with computing platform 114 as user interface 116 include, but are not limited to, an RS-232 port, RF link, an IR link, modem (telephone, cable or other). In short, any technique for communicating information with computing platform 114 and/or system 100 is contemplated by the present disclosure as user interface 116.

Electronic storage 118 may include electronic storage media that electronically stores information. The electronic storage media of electronic storage 118 may include one or both of system storage that is provided integrally (i.e., substantially non-removable) with computing platform 114 and/or removable storage that is removably connectable to computing platform 114 via, for example, a port (e.g., a USB port, a firewire port) or a drive (e.g., a disk drive). Electronic storage 118 may include one or more of optically readable storage media (e.g., optical disks), magnetically readable storage media (e.g., magnetic tape, magnetic hard drive, floppy drive), electrical charge-based storage media (e.g., EEPROM, RAM), solid-state storage media (e.g., flash drive), and/or other electronically readable storage media. Electronic storage 118 may include one or more virtual storage resources (e.g., cloud storage, a virtual private network, and/or other virtual storage resources). Electronic storage 118 may store software algorithms, information determined by processor 106, information received from external resources 120, information entered and/or selected via user interface 116, and/or other information that enables system 100 to function as described herein.

External resources 120 include sources of information such as databases, websites, etc.; external entities participating with system 100 (e.g., systems or networks that store data associated with the cancer patient), one or more servers outside of system 100, a network (e.g., the internet), electronic storage, equipment related to Wi-Fi™ technology, equipment related to Bluetooth® technology, data entry devices, or other resources. In this disclosure, some or all of the functionality attributed herein to external resources 120 may be provided by resources included in system 100. External resources 120 may be configured to communicate with computing platform 114, physical activity sensor 104, body position sensor 102, and/or other components of system 100 via wired and/or wireless connections, via a network (e.g., a local area network and/or the internet), via cellular technology, via Wi-Fi technology, and/or via other resources.

Body position sensor 102, physical activity sensor 104, computing platform 114, and/or external resources 120 may be operatively linked via one or more electronic communication links. For example, such electronic communication links may be established, at least in part, via wires, via local network using Wi-Fi, Bluetooth, and/or other technologies, via a network such as the Internet and/or a cellular network, and/or via other networks. It will be appreciated that this is not intended to be limiting, and that the scope of this disclosure includes embodiments in which body position sensor 102, physical activity sensor 104, computing platform 114, and/or external resources 120 may be operatively linked via some other communication media, or with linkages not shown in FIG. 1. In this disclosure, as described above, computing platform 114, body position sensor 102, physical activity sensor 104, and/or other devices may be integrated as a singular device.

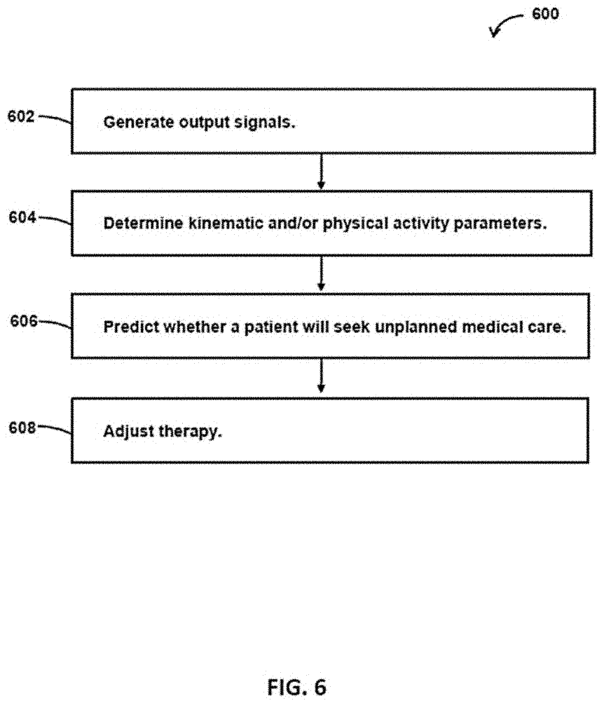

FIG. 6 illustrates a method 600 for determining whether a cancer patient will need unplanned medical care during cancer therapy with a determination system, in accordance with one or more embodiments. Unplanned medical care may comprise medical care unrelated to the cancer therapy, unscheduled medical care, non-routine medical care, emergency medical care, and/or other unplanned medical care. The system comprises one or more sensors, one or more processors, and/or other components. The operations of method 600 presented below are intended to be illustrative. In this disclosure, method 600 may be accomplished with one or more additional operations not described, and/or without one or more of the operations discussed. Additionally, the order in which the operations of method 600 are illustrated in FIG. 6 and described below is not intended to be limiting.

In this disclosure, method 600 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 600 in response to instructions stored electronically on an electronic storage medium. The one or more processing devices may include one or more devices configured through hardware, firmware, and/or software to be specifically designed for execution of one or more of the operations of method 600.

At an operation 602, output signals may be generated. In this disclosure, the output signals may convey spatial position information related to spatial positions of one or more anatomical sites on the cancer patient while the cancer patient performs a prescribed movement. The spatial position information may comprise visual information representing the body of the cancer patient and/or other information. The one or more anatomical sites may comprise an anatomical site that corresponds to a center of mass of the cancer patient. In this disclosure, the one or more anatomical sites may comprise anatomical sites indicative of mobility and/or the center of mass of a cancer patient, and/or other anatomical sites. In this disclosure, a location that corresponds to the center of mass and/or that is indicative of mobility may be a location at a base of a spine of the cancer patient, a location at or near the hips of a cancer patient, locations and/or near the knees of a cancer patient, and/or other locations. The prescribed movement may comprise movement associated with a chair to table (CTT) exam and/or other movement, for example.

In this disclosure, the output signals may convey physical activity information related to physical activity performed by the cancer patient. In these embodiments, the one or more sensors may comprise a wrist worn motion sensor and/or other sensors, for example. In this disclosure, operation 602 may be performed by one or more sensors similar to or the same as body position sensor 102 and/or physical activity sensor 104 (shown in FIG. 1, and described herein).

At an operation 604, kinematic and/or physical activity parameters may be determined. In this disclosure, the one or more determined kinematic and/or physical activity parameters may be features extracted from the spatial position or physical activity information, and/or other parameters. In this disclosure, the determined kinematic and/or physical activity parameters may comprise less bytes of data than the spatial position information and/or the physical activity information conveyed by the one or more output signals. In this disclosure, operation 604 may include determining one or more kinematic parameters indicative of the movement of the cancer patient during the prescribed movement based on the spatial position information and/or other information. The one or more kinematic parameters may comprise velocities, accelerations, and/or other kinematic parameters. For example, the one or more kinematic parameters may comprise an acceleration of an anatomical site that corresponds to the center of mass of the cancer patient, a velocity and/or acceleration of an anatomical site indicative of mobility of the cancer patient, and/or other parameters. In this disclosure, determining the one or more kinematic parameters indicative of the movement of the cancer patient during the prescribed movement based on the spatial position information comprises determining anatomical site position vectors for the one or more anatomical sites. The anatomical site position vectors may comprise three-dimensional time series generated for given positions of the one or more anatomical sites at given time points during the prescribed movement. This may also include determining accelerations for the one or more anatomical sites based on the anatomical site position vectors using a mean-value theorem. The acceleration of an anatomical site that corresponds to the center of mass (for example) of the cancer patient may be determined using the mean-value theorem based on anatomical site position vectors for the anatomical site that corresponds to the center of mass of the cancer patient, for example.

In this disclosure, operation 604 may include determining one or more physical activity parameters indicative of the physical activity of the cancer patient based on the physical activity information and/or other information. In these embodiments, the one or more physical activity parameters may comprise metabolic equivalence (METs) and/or other parameters. In this disclosure, operation 604 may be performed by one or more processors configured to execute a computer program component similar to or the same as parameter component 112 (shown in FIG. 1, and described herein).

Operation 606 may include determining whether a patient will need unplanned medical care. In this disclosure, the determining may be based on an acceleration of an anatomical site that corresponds to the center of mass of the cancer patient, velocities and/or accelerations of anatomical sites indicative of mobility, and/or other information. In this disclosure, the determining may be based on the metabolic equivalence determined for the cancer patient, and/or other information.

In this disclosure, the determination of whether the cancer patient will need unplanned medical care during cancer therapy is indicative of a future reaction of the cancer patient to chemotherapy and/or radiation during cancer therapy. In this disclosure, determining whether the cancer patient will need unplanned medical care during cancer therapy comprises determining whether the cancer patient will need unplanned medical care during a future period of time that corresponds to one or more cancer therapy treatments received by the cancer patient. In this disclosure, the future period of time is about two months and/or other periods of time. In this disclosure, operation 606 comprises categorizing the cancer patient as either likely to likely to need unplanned medical care or unlikely to need unplanned medical care during cancer therapy. In this disclosure, operation 606 comprises determining a likelihood the cancer patient will need unplanned medical care, and categorizing the cancer patient into two or more groups based on the likelihood. In this disclosure, operation 606 may be performed by one or more processors configured to execute a computer program component similar to or the same as determination component 113 (shown in FIG. 1, and described herein).

At an operation 608, therapy may be adjusted. The adjusted therapy may be the cancer therapy and/or other therapies. The adjusting may be based on the determination of whether the patient will need unplanned medical care and/or other information. In this disclosure, adjusting may include facilitating adjustment of the cancer therapy based on the determination of whether the cancer patient will need unplanned medical care during cancer therapy. In this disclosure, facilitating may comprise determining and displaying recommended changes, determining one or more additional parameters from the information in the output signals from the one or more sensors, and/or other operations. In this disclosure, operation 608 may be performed by one or more processors configured to execute a computer program component similar to or the same as determination component 113 (shown in FIG. 1 and described herein).

### Methods

Trial Design.

This study was a multicenter, single arm, observational trial conducted in the United States. Kinematic signatures obtained from motion-capture systems (e.g. Microsoft Kinect) and wearable motion sensors (e.g. Microsoft Band) were correlated with unexpected hospital visits and physical activity at home. The institutional review boards at all participating sites approved the study protocol. Written informed consent was obtained from all participants.

Participants.

Briefly, patients were eligible for the study if they were >18 years of age, had a diagnosis of a solid tumor, and undergoing two planned cycles of highly emetogenic chemotherapy, could ambulate without an assistive device, and had 2 separate kinematic evaluations successfully completed.

TABLE 1
Baseline Characteristics of Participants.

| | |
|---|---|
| Number of patients | 36 |

Age

| | |
|---|---|
| Median | 48 |
| Range | 24-72 |

Gender

| | |
|---|---|
| Male | 18 |
| Female | 18 |

Ethnicity

| | |
|---|---|
| Hispanic | 22 |
| Non-Hispanic | 14 |

Goal of treatment

| | |
|---|---|
| Curative | 30 |
| Palliative | 6 |

Clinical Exercises and Motion Capture.

Patients underwent two clinically supervised tasks including chair-to-table (CTT) and get-up-and-walk (GUP). CTT task begins with patients standing up from a chair while rotating the hip and left leg and pivoting on the right leg. Therefore, the CTT task design requires larger range of motion from the left lower extremities. The GUP task requires patients to stand up and walk to a marker 8 feet away, turn, and walk back to the starting position. We analyze the entire CTT task and the walking portion of GUP using the motion capture system.

The two tasks are performed by the cohort of cancer patients once pre-treatment (visit-1) and once post-treatment (visit-2). The Microsoft Kinect, a depth-sensing motion capture camera is used record the exercises, and three-dimensional positions of 25 anatomical sites (FIG. 3) are extracted, from which six types of kinematic features are calculated: 1) velocity, 2) acceleration, 3) specific kinetic energy, 4) specific potential energy, 5) sagittal angle, 6) angular velocity. We exclude wrist, hand, ankle, and foot joints (FIG. 3) from statistical analysis as the motion capture signal for these joints is less reliable. The combination of selected joints and kinematic features capture the underlying biomechanics of patient movement and are therefore selected for inter-patient comparison.

Each patient has a pre- and post-treatment pair of samples of each feature, and four statistics (minimum, maximum, mean, median) from each visit's time series kinematic feature are averaged (mean) over the two samples. Hereafter, we refer to the mean-(minimum, maximum, mean, median) over the two visits simply as the minimum, maximum, mean, and median.

Physical Activity Measure.

Patient outcomes were grouped by activity level and unexpected hospital visits. During the study period that spanned for 60 days while receiving chemotherapy and a 90-day follow-up period, patients wore a wrist motion sensor to track their overall daily physical activity. We recorded the number of hours spent above low physical activity (LPA) for each patient over this period. Patients were considered high activity, rather than low activity, if they met greater than a 15-hour physical activity threshold. Patients with more than 15 hours of activity above LPA (HALPA=0) and patients with 15 hours or less active time than LPA form the two HALPA groups.

Likewise, patients were grouped if they had one or more unexpected hospital visits compared to those that did not have any. Four types of unexpected hospital visits were tracked including: 1) Unplanned triage/infusion center visits, 2) urgent office visits, 3) urgent hospitalizations, and 4) ER visits. Patients with zero unexpected hospitalizations (UHV=0) and patients with one or more unexpected hospital visits are (UHV=1) form the UHV groups.

Statistical Analysis.

Patients were differentiated by the average of visit-1 and visit-2 statistics for the set of kinematic features and correlate to two binarized clinical outcome UHV and HALPA. The Welch's t-test is used to test whether the mean value of the four averaged statistics is different for the UHV or HALPA groups, thereby revealing kinematic features which distinguish between UHV=0 and UHV=1 patients, and similarly HALPA=0 and HALPA=1 patients. The Welch's t-test also known as the unequal variance t-test allows the central tendency of two groups of unequal sizes and unequal variance to be tested for equivalence. Secondly, we calculate the receiver operating characteristic (ROC) curve and use the corresponding area under the curve (AUC) as a metric of a feature's ability to classify patients into risk groups.

Patient Cohort/Enrollment Criteria

Of the 60 persons screened and agreed to participate in the study, 36 persons completed the study without drop out and had associated unexpected hospital visits and physical activity results. Overall the mean age of participates were 47.8 years old, and 50% were men. Breast, testicular, and head and neck cancer, comprised most of study participants. Chemotherapy was primarily of curative intent for most patients. Presumed reasons for higher than expected study drop out were likely due to a large proportion of persons being recruited from the Los Angeles County Hospital uninsured patient population combined with a large proportion being young males receiving chemotherapy for testicular cancer. These factors may explain why there was not a higher percentage of patients could complete the five-month study period.

There are 16 UHV=0 patients and 20 UHV=1 patients for a total of N=36 patients for whom hospitalization data is collected. Similarly, there are 17 HALPA=0 patients and 18 HALPA=1 patients for a total of N=35 patients for whom physical activity data is collected.

Unexpected Hospitalizations.

The kinematic features that correlate most with unexpected hospital visits were reported according to i) t-test and ii) ROC analysis in Table 2. CTT features dominate the list of UHV differentiating kinematic features and GUP features were less associated with the two outcomes. The full list of 55 features with significant t-test scores (p-value <0.05) are listed below.

TABLE 2
Top ten kinematic features from Welch's t-test (ranked by
absolute value of two-sample t-test scores) and top ten kinematic
features with highest AUC for differentiating between patients with
no unexpected hospitalizations (UHV = 0) and patients with one
or more unexpected hospitalizations (UHV = 1).

| Welch's t-test | | | | ROC analysis | | |
|---|---|---|---|---|---|---|
| | Feature | t-test | p-value | | Feature | AUC |
| 1 | Left knee: mean CTT acc | 3.735 | 0.001 | 1 | Left leg: max CTT av-y | 0.816 |
| 2 | Left hip: mean CTT acc | 3.398 | 0.002 | 2 | Left knee: mean CTT acc | 0.806 |
| 3 | Spine base: mean CTT acc | 3.258 | 0.003 | 3 | Left elbow: max CTT pe | 0.781 |
| 4 | Left knee: mean CTT vel | 3.177 | 0.003 | 4 | Left hip: max CTT acc | 0.781 |
| 5 | Left knee: mean CTT ke | 3.14 | 0.004 | 5 | Spine base: mean CTT acc | 0.775 |
| 6 | Left elbow: max CTT pe | 2.988 | 0.005 | 6 | Left hip: mean CTT acc | 0.775 |
| 7 | Right hip: mean CTT acc | 2.928 | 0.006 | 7 | Left knee: mean CTT ke | 0.775 |
| 8 | Left hip: max CTT acc | 2.925 | 0.006 | 8 | Right leg: min CTT av-x | 0.759 |
| 9 | Left hip: mean CTT ke | 2.921 | 0.006 | 9 | Hip: min CTT av-z | 0.756 |
| 10 | Right arm: mean GUP av-y | 2.91 | 0.006 | 10 | Left hip: mean CTT ke | 0.753 |

(vel: velocity; acc: acceleration; pe: potential energy; ke: kinetic energy; sa: sagittal angle; av-x, av-y, av-z: angular velocity about x, y, or z axes).

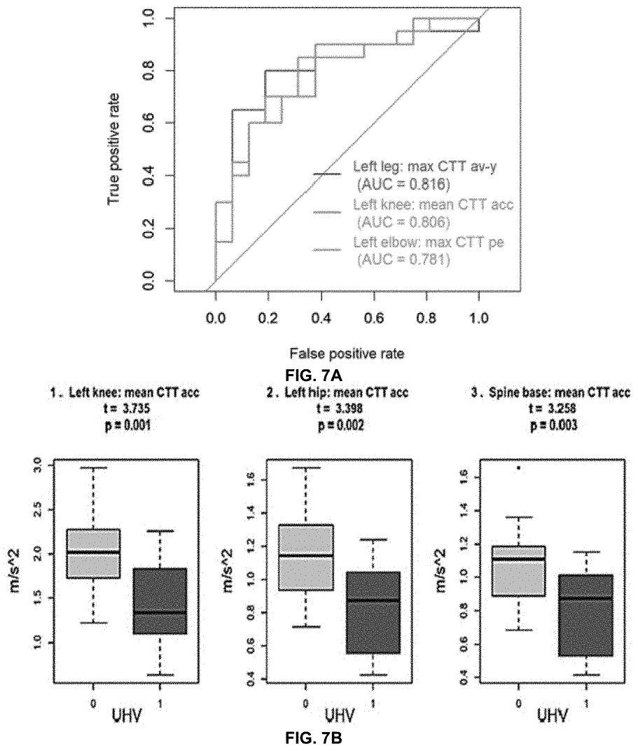

Hip and left side joints are the top UHV features due to the pivot on the right side, and resulting large left side motion of CTT (FIG. 3). FIG. 7A shows the ROC curves for the features with the highest AUC values for UHV where the maximum left leg angular velocity about the y-axis during CTT forms the best classifier of UHV (AUC=0.816). The top three UHV differentiating features according to the t-test are plotted in FIG. 7B, which shows the left knee, left hip, and the spine base mean accelerations during CTT are all generally higher for patients with no unexpected hospitalizations compared to patients with one or more unexpected hospitalizations.

Physical Activity.

Kinematic features that correlate most with physical activity according to i) t-test and ii) ROC analysis in Table 3. Unlike UHV, both CTT and GUP features appear in the list of HALPA differentiating kinematic features. The full list of 15 features with significant t-test scores (p-value <0.05) are listed in Appendix D. Angular velocities, particularly those of the hip, differentiate HALPA groups the most. Nevertheless, kinematic features from the clinical exercises are less correlated with HALPA groups than UHV groups as both t-test scores and AUC values are generally lower in Table 3 compared to Table 2.

TABLE 3
Top ten kinematic features from Welch's t-test (ranked by absolute value of
two-sample t-test scores) and top ten kinematic features with highest AUC for
differentiating between patients with more than 15 hours of activity above LPA
(HALPA = 0) and patients with 15 hours or less activity above LPA (HALPA = 1).

| Welch's t-test | | | | ROC analysis | | |
|---|---|---|---|---|---|---|
| | Feature | t-test | p-value | | Feature | AUC |
| 1 | Hip: mean GUP av-x | −2.414 | 0.022 | 1 | Hip: mean CTT av-z | 0.735 |
| 2 | Left leg: min GUP av-x | −2.379 | 0.024 | 2 | Hip: mean CTT av-y | 0.729 |
| 3 | Back: mean CTT sa | −2.331 | 0.026 | 3 | Left arm: mean GUP av-y | 0.725 |
| 4 | Left arm: min GUP av-y | −2.328 | 0.032 | 4 | Left knee: median GUP ke | 0.722 |
| 5 | Right leg: mean GUP av-z | 2.224 | 0.033 | 5 | Left leg: min GUP av-x | 0.722 |
| 6 | Left hip: mean CTT acc | 2.221 | 0.033 | 6 | Spine mid: mean CTT acc | 0.719 |
| 7 | Back: median CTT sa | −2.219 | 0.034 | 7 | Right leg: median CTT av-y | 0.719 |
| 8 | Hip: mean CTT av-x | −2.193 | 0.035 | 8 | Back: mean CTT sa | 0.716 |
| 9 | Left knee: median GUP ke | 2.185 | 0.039 | 9 | Shoulder: median CTT av-x | 0.712 |
| 10 | Right leg: median CTT av-y | −2.184 | 0.037 | 10 | Hip: mean CTT av-x | 0.706 |

(vel: velocity; acc: acceleration; pe: potential energy; ke: kinetic energy; sa: sagittal angle; av-x, av-y, av-z: angular velocity about x, y, or z axes).

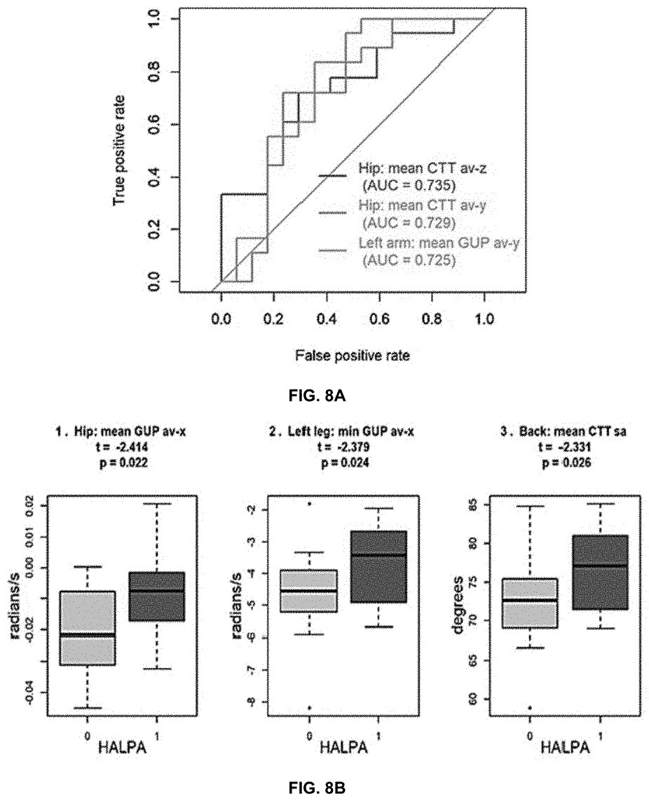

FIG. 8A shows the ROC curves for the features with the highest AUC values for HALPA where the mean hip angular velocity about the vertical axis during CTT forms the best classifier of HALPA (AUC=0.735). Mean hip and minimum left leg angular velocities during GUP are both larger (absolute value) for higher activity patients as seen in FIG. 8B.

### Example 1. Calculating the Emetogenicity of Multiple Agent Chemotherapy and/or Biotherapy Regimens

The information in Table 4 was used to calculate the emetogenicity of multiple agent chemotherapy/biotherapy regimens.

Step and guidelines for these calculations are as follows: First, list each agent contained within the multiple agent regimen, then identify the agent with the highest emetogenic level, and finally determine the contribution of the remaining agents using the following guidelines.

Guideline 1. Level 1 agents do not contributor to emetogenicity in combination regimens. For example, Level 1+1=0, 2+1=2, 3+1=3, and 4+1=4.

Guideline 2. Adding one or more level 2 agents increases the highest level by 1 in combination regimens. For example, Level 2+2=3, 3+2=4, and 2+2+2=3 3+2+2=4.

Guideline 3. Adding level 3 or 4 agents increase the highest level by 1 per each agent in combination regimens. For example, Level 3+3=4, 3+3+3=5, and 4+3=5.

TABLE 4
Chemotherapy Emetogenicity Table.

[symbol]

Emetogenic·Risk·of·Chemotherapy·and·Biotherapy·Agents·

| | Agents· |
|---|---|
| Emetogenicity· | (alphabetically) |
| 5· | AC·combo:·doxorubicin·or·epirubicin·+·cyclophosphamide· |
| 1· | Alemtuzumab· |
| 1· | Alpha·Interferon·<·5000·IU/m2· |
| 2· | Amifostine·<·300·mg· |
| 4· | Amifostine·≥·300[]500·mg/m2· |
| 1· | Androgens· |
| 3· | Arsenic·trioxide· |
| 1· | Asparaginase· |
| 3· | Azacitadine· |
| 3· | Bendamustine· |
| 1· | Bevacizumab· |
| 2· | Bexarotene·(oral)· |
| 1· | Bleomycin· |
| 1· | Bortezomib· |
| 4· | Busulfan·>·4·mg/m2· |
| 2· | Capecitabine·(oral)· |
| 4· | Carboplatin· |
| 4· | Carmustine·[]·250·mg/m2· |
| 5· | Carmustine·>·250·mg/m2· |
| 1· | Cetuximab· |
| 1· | Chloambucil·(oral)· |
| 5· | Cisplatin·[]50·mg/m2· |
| 4· | Cisplatin·<·50·mg/m2· |
| 1· | Cladribine· |
| 3· | Clofarabine· |
| 1· | Corticosteroids· |
| 3· | Cyclophosphamide·(oral)· |
| 3· | Cyclophosphamide·[]·750·mg/m2· |
| 4· | Cyclophosphamide·>·750·mg/m2·to·[]·1,500·mg/m2· |
| 5· | Cyclophosphamide·>·1,500·mg/m2· |
| 2· | Cytarabine·(low·dose)·100[]200·mg/m2· |
| 4· | Cytarabine·>·1·g/m2· |
| 5· | Dacarbazine· |
| 4· | Dactinomycin· |
| 1· | Dasatinib·(oral)· |
| 3· | Daunorubicin· |
| 1· | Denileukin diftitox· |
| 1· | Dexazoxane· |
| 2· | Docetaxel· |
| 2· | Doxorubicin·(liposomal)· |
| 3· | Doxorubicin·<·60·mg/m2· |
| 4· | Doxorubicin·≥·60·mg/m2· |
| 3· | Epirubicin·[]·90·mg/m2· |
| 4· | Epirubicin·>·90·mg/m2· |
| 1· | Erlotinib·(oral)· |
| 2· | Etoposide· |
| 2· | Fluorouracil· |
| 1/2· | Fludarabine· |
| 2· | Gemcitabine· |
| 1· | Gemtuzumab ozogamicin· |
| 1· | Gefitinib·(oral)· |
| 3· | Hexamethylmelamine·(oral)· |
| 1· | Hydroxyurea·(oral)· |
| 1· | Ibritumomab·tiuxetan· |
| 3· | Isosfamide· |
| 1· | Imatinib·mesylate·(oral)· |
| 3· | Interlukin[]2·>·12[]15·million·units/m2· |
| 3· | Irinotecan· |
| 1· | Ixabepilone· |
| 1· | Lapatinib·(oral)· |
| 2· | Lenalidomide· |
| 3· | Lomustine·(oral)· |
| 5· | Mechorethamine· |
| 1· | Melphalan·(oral·low[]does)· |
| 4· | Melphalan·>·50·mg/m2· |
| 1· | Methotrexate·[]·50·mg/m2· |
| 2· | Methotrexate·>·50·mg/m2·<·250·mg/m2· |
| 3· | Methotrexate·250[]1,000·mg/m2· |
| 4· | Methotrexate·>·1,000·mg/m2· |
| 2· | Mitomicin· |
| 2· | Mitoxantrone·<·15·mg/m2· |
| 1· | Nelarabine· |
| 3· | Oxaliplatin·>·75·mg/m2· |
| 2· | Placlitaxel/Placlitaxel·albumin[]bound· |
| 1· | Panitumumab· |
| 2· | Pemetrexed· |
| 1· | Pentostatin· |
| 5· | Procarbazine·(oral)· |
| 1· | Rituximab· |
| 2· | Sorafenib·(oral)· |
| 5· | Streptozocin· |
| 2· | Sunitinib·(oral)· |
| 3· | Temozolomide·(oral)· |
| 1· | Temsirolimus· |
| 2· | Teniposide· |
| 1· | Thioguanine·(oral)· |
| 2· | Topotecan· |
| 1· | Tositumomab· |
| 1· | Trastuzumab· |
| 1· | Trentinoin·(oral)· |
| 1· | Vinblastine· |
| 1· | Vincristine· |
| 1· | Vinorelbine· |
| 3· | Vinorelbine·(oral)· |
| 2· | Vorinostat·(oral)· |

NCCN·Levels·of·Emetogenicity:·
Level·5·-·High·Emetic·Risk:·90%·frequency·of·emesis·
Level·3/4·-·Moderate·Emetic·Risk:·30[]90%·frequency·of·emesis·
Level·2·-·Low·Emetic·Risk:·10[]30%·frequency·of·emesis·
Level·1·-Minimal·Emetic·Risk:·<10%·frequency·of·emesis·

Kinematic Feature Extraction.

Details of kinematic feature extraction from the raw three-dimensional position motion capture data are described here. Anatomical site position vectors [symbol] = [symbol] are three-dimensional time series constructed from position at each time point, rᵢ(t)=(xᵢ(t), yᵢ(t), zᵢ(t)) for i=25 anatomical sites. The position vectors are used to calculate velocity magnitude, [symbol] ᵢ=( [symbol] ^{T} [symbol] + [symbol] ^{T} [symbol] + [symbol] ^{T} [symbol] )^{1/2} and acceleration magnitude \vec{α}ᵢ=( [symbol] + [symbol] + [symbol] )^{1/2} of each anatomical site using the mean-value theorem. Due to the lack of distribution of mass information, specific kinetic energy [symbol] =½ [symbol] ᵢ^{T} [symbol] ᵢ and specific potential energy [symbol] =gΔ [symbol] ᵢ=g( [symbol] ᵢ− [symbol] ᵢ(t=t₁). We define sagittal angle as the angle formed between [symbol] _{1,m} the vector originating at the spine base and pointing in the direction of motion, and [symbol] _{1,3} the vector connecting anatomical site 1 (spine base) and 3 (neck) at each time point. The angular velocity of the sections defined in FIG. 1 are calculated using three-dimensional rigid body kinematic equations for relative motion.

Sagittal Angle Calculation.

We define sagittal angle as the angle formed between [symbol] _{1,m} the vector originating at the spine base and pointing in the direction of motion, and [symbol] _{1,3} the vector connecting anatomical site 1 (spine base) and 3 (neck) at each time point. The sagittal angle is calculated using the inverse tangent of the ratio of the cross product and dot product of [symbol] _{1,m} and [symbol] _{1,3}, θₛ=tan⁻¹(∥ [symbol] _{1,m}× [symbol] _{1,3}∥/ [symbol] _{1,m}· [symbol] _{1,3}).

Angular Velocity Calculation.

The angular velocity of the sections defined in FIG. 1 are calculated using three-dimensional rigid body kinematic equations for relative motion. A section (FIG. 1) is treated as a rigid bar and is defined by two anatomical points (e.g. left and right hips define the hip section) and we refer generically to these two ends as point A and point B. We calculate the velocities of these two points from the position vectors using the mean-value theorem as mentioned previously. Therefore, using these two velocities, the angular velocity of the section \vec{ω}_{AB} can be isolated in the relative velocity vector equation, [symbol] _{B}− [symbol] _{A}=\vec{ω}_{AB}× [symbol] =(Δvₓ, Δv_{y}, Δv_{z}) where [symbol] is the vector from point A to point B [symbol] =r_{B}−r_{A}=(r_{AB,x}, r_{AB,y}r_{AB,z}). This vector equation has three components corresponding to the three directions and require an additional equation to solve for the three components of the angular velocity. Consequently, we use a kinematic restriction equation [symbol] · [symbol] =0, because the angular motion of the section along the axis of the section does not affect its action. This allows for a solution to the three components of the angular velocity vector [symbol] =(ωₓ, ω_{y}, ω_{z}):

$${ω}_{x} = \frac{Δ {v}_{z} {r}_{AB , y} - Δ {v}_{y} {r}_{AB , z}}{{r}_{AB , x}^{2} + {r}_{AB , y}^{2} + {r}_{AB , z}^{2}}$$

$${ω}_{y} = \frac{1}{{r}_{AB , x}} ( {r}_{AB , y} {ω}_{x} - Δ {v}_{z} )$$

$${ω}_{z} = \frac{1}{{r}_{AB , y}} ( {r}_{AB , z} {ω}_{y} - Δ {v}_{x} )$$

These equations are solved at each time point to get the time series of angular velocities for each section in FIG. 3.

Two-Sample t-Tests.

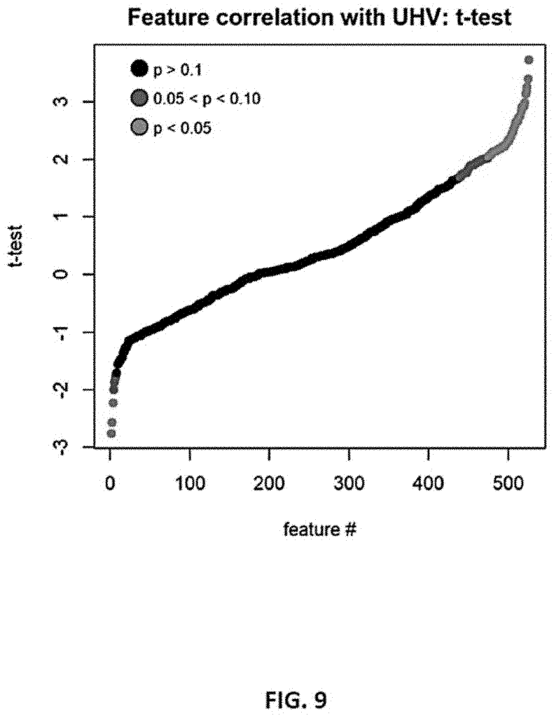

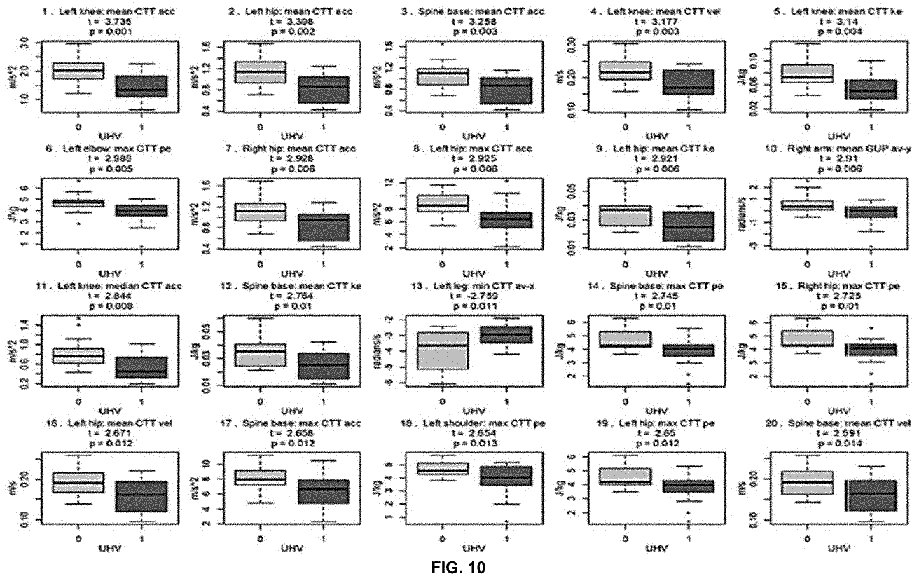

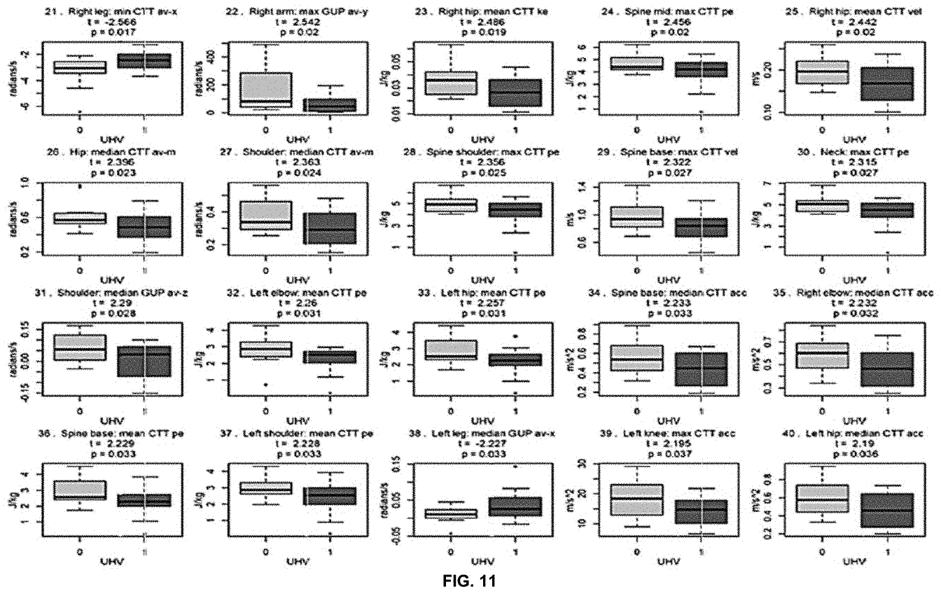

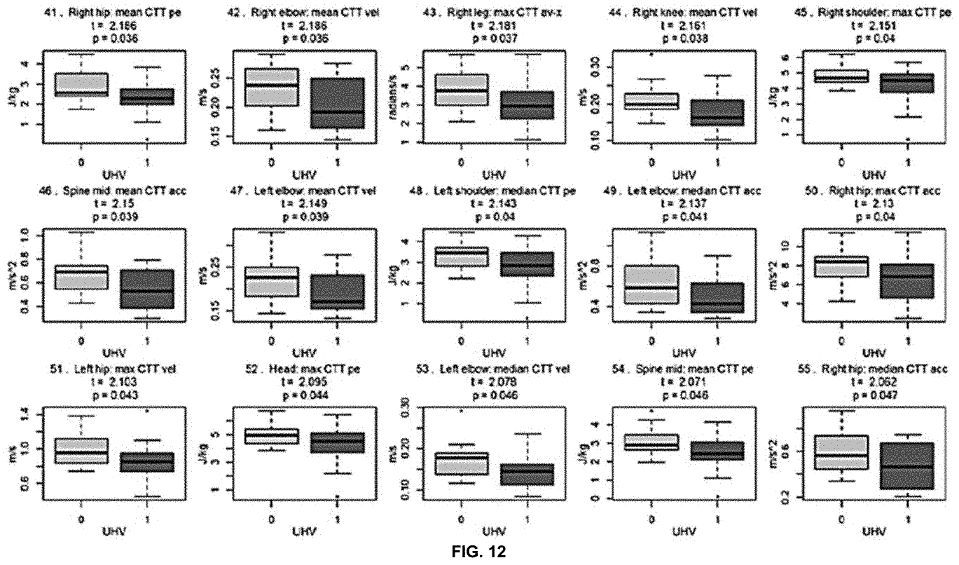

Two-sample t-tests are done to determine if mean values of kinematic features are different for patients with zero unexpected hospitalizations (UHV=0) and patients with one or more hospitalizations (UHV=1), and the distribution of the resulting t-test scores and significance values for the entire set of 526 features is shown in FIG. 9. Full list of 55 significant (p-value <0.05) t-test scores is shown in Table 5, and boxplots of these significantly differentiating kinematic features is shown in FIGS. 10-12.

TABLE 5
Full list of kinematic features which significantly (p-value <
0.05) differentiate between patients with no unexpected hospitalizations
(UHV = 0) and patients with one or more unexpected hospitalizations
(UHV = 1). Ranked by absolute value of two-sample t-test scores.

| | Feature | t-test | p-value |
|---|---|---|---|
| 1 | Left knee: mean CTT acc | 3.735 | 0.001 |
| 2 | Left hip: mean CTT acc | 3.398 | 0.002 |
| 3 | Spine base: mean CTT acc | 3.258 | 0.003 |
| 4 | Left knee: mean CTT vel | 3.177 | 0.003 |
| 5 | Left knee: mean CTT ke | 3.14 | 0.004 |
| 6 | Left elbow: max CTT pe | 2.988 | 0.005 |
| 7 | Right hip: mean CTT acc | 2.928 | 0.006 |
| 8 | Left hip: max CTT acc | 2.925 | 0.006 |
| 9 | Left hip: mean CTT ke | 2.921 | 0.006 |
| 10 | Right arm: mean GUP av-y | 2.91 | 0.006 |
| 11 | Left knee: median CTT acc | 2.844 | 0.008 |
| 12 | Spine base: mean CTT ke | 2.764 | 0.01 |
| 13 | Left leg: min CTT av-x | −2.759 | 0.011 |
| 14 | Spine base: max CTT pe | 2.745 | 0.01 |
| 15 | Right hip: max CTT pe | 2.725 | 0.01 |
| 16 | Left hip: mean CTT vel | 2.671 | 0.012 |
| 17 | Spine base: max CTT acc | 2.658 | 0.012 |
| 18 | Left shoulder: max CTT pe | 2.654 | 0.013 |
| 19 | Left hip: max CTT pe | 2.65 | 0.012 |
| 20 | Spine base: mean CTT vel | 2.591 | 0.014 |
| 21 | Right leg: min CTT av-x | −2.566 | 0.017 |
| 22 | Right arm: max GUP av-y | 2.542 | 0.02 |
| 23 | Right hip: mean CTT ke | 2.486 | 0.019 |
| 24 | Spine mid: max CTT pe | 2.456 | 0.02 |
| 25 | Right hip: mean CTT vel | 2.442 | 0.02 |
| 26 | Hip: median CTT av-m | 2.396 | 0.023 |
| 27 | Shoulder: median CTT av-m | 2.363 | 0.024 |
| 28 | Spine shoulder: max CTT pe | 2.356 | 0.025 |
| 29 | Spine base: max CTT vel | 2.322 | 0.027 |
| 30 | Neck: max CTT pe | 2.315 | 0.027 |
| 31 | Shoulder: median GUP av-z | 2.29 | 0.028 |
| 32 | Left elbow: mean CTT pe | 2.26 | 0.031 |
| 33 | Left hip: mean CTT pe | 2.257 | 0.031 |
| 34 | Spine base: median CTT acc | 2.233 | 0.033 |
| 35 | Right elbow: median CTT acc | 2.232 | 0.032 |
| 36 | Spine base: mean CTT pe | 2.229 | 0.033 |
| 37 | Left shoulder: mean CTT pe | 2.228 | 0.033 |
| 38 | Left leg: median GUP av-x | −2.227 | 0.033 |
| 39 | Left knee: max CTT acc | 2.195 | 0.037 |
| 40 | Left hip: median CTT acc | 2.19 | 0.036 |
| 41 | Right hip: mean CTT pe | 2.186 | 0.036 |
| 42 | Right elbow: mean CTT vel | 2.186 | 0.036 |
| 43 | Right leg: max CTT av-x | 2.181 | 0.037 |
| 44 | Right knee: mean CTT vel | 2.161 | 0.038 |
| 45 | Right shoulder: max CTT pe | 2.151 | 0.04 |
| 46 | Spine mid: mean CTT acc | 2.15 | 0.039 |
| 47 | Left elbow: mean CTT vel | 2.149 | 0.039 |
| 48 | Left shoulder: median CTT pe | 2.143 | 0.04 |
| 49 | Left elbow: median CTT acc | 2.137 | 0.041 |
| 50 | Right hip: max CTT acc | 2.13 | 0.04 |
| 51 | Left hip: max CTT vel | 2.103 | 0.043 |
| 52 | Head: max CTT pe | 2.095 | 0.044 |
| 53 | Left elbow: median CTT vel | 2.078 | 0.046 |
| 54 | Spine mid: mean CTT pe | 2.071 | 0.046 |
| 55 | Right hip: median CTT acc | 2.062 | 0.047 |

(vel: velocity; acc: acceleration; pe: potential energy; ke: kinetic energy; sa: sagittal angle; av-x, av-y, av-z: angular velocity about x, y, or z axes).

Two-Sample t-Tests

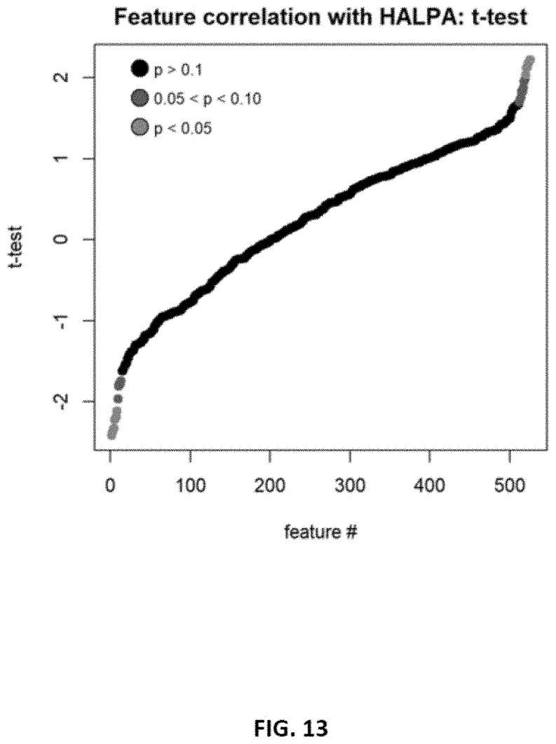

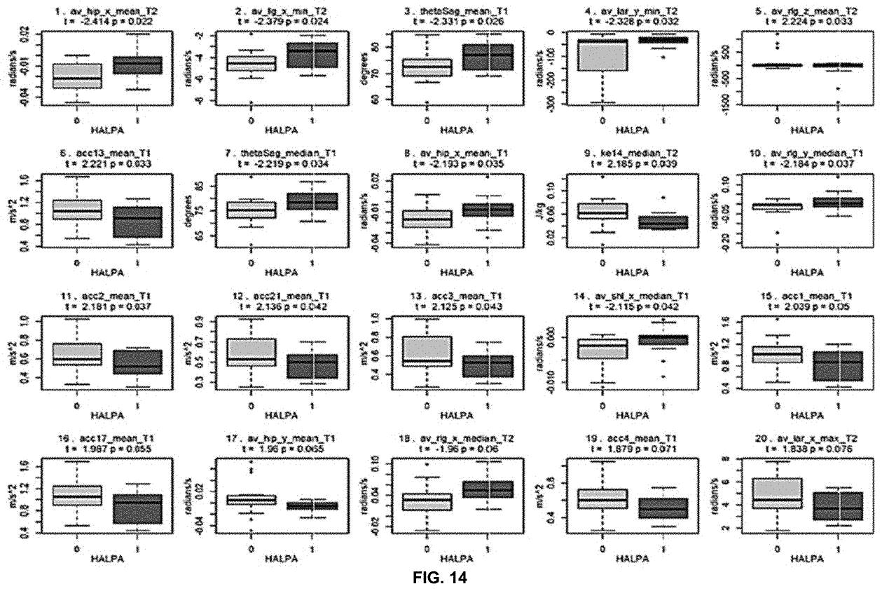

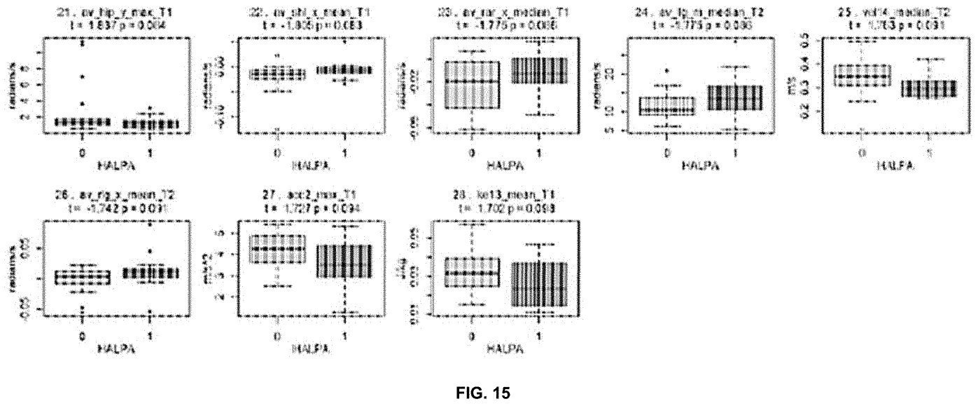

Two-sample t-tests are done to determine if mean values of kinematic features are different for patients with 15 hours or more of activity above LPA (HALPA=0) from patients with 15 hours or less of activity above LPA (HALPA=1), and the distribution of the resulting t-test scores and significance values for the entire set of 526 features is shown in FIG. 13. Full list of 28 significant (p-value <0.05) t-test scores is shown in Table 6, and boxplots of these significantly differentiating kinematic features is shown in FIGS. 14-15.

TABLE 6
Full list of kinematic features which (feature 1-15: p-value < 0.05,
feature 16-28: 0.05 < p-value < 0.10) differentiate between patients with
no unexpected hospitalizations (UHV = 0) and patients with one or more
unexpected hospitalizations (UHV = 1). Ranked by absolute
value of two-sample t-test scores.

| | Feature | t-test | p-value |
|---|---|---|---|
| 1 | Hip: mean GUP av-x | −2.414 | 0.022 |
| 2 | Left leg: min GUP av-x | −2.379 | 0.024 |
| 3 | Back: mean CTT sa | −2.331 | 0.026 |
| 4 | Left arm: min GUP av-y | −2.328 | 0.032 |
| 5 | Right leg: mean GUP av-z | 2.224 | 0.033 |
| 6 | Left hip: mean CTT acc | 2.221 | 0.033 |
| 7 | Back: median CTT sa | −2.219 | 0.034 |
| 8 | Hip: mean CTT av-x | −2.193 | 0.035 |
| 9 | Left knee: median GUP ke | 2.185 | 0.039 |
| 10 | Right leg: median CTT av-y | −2.184 | 0.037 |
| 11 | Spine mid: mean CTT acc | 2.181 | 0.037 |
| 12 | Spine shoulder: mean CTT acc | 2.136 | 0.042 |
| 13 | Neck: mean CTT acc | 2.125 | 0.043 |
| 14 | Shoulder: median CTT av-x | −2.115 | 0.042 |
| 15 | Spine base: mean CTT acc | 2.039 | 0.05 |
| 16 | Right hip: mean CTT acc | 1.987 | 0.055 |
| 17 | Hip: mean CTT av-y | 1.96 | 0.065 |
| 18 | Right leg: median GUP av-x | −1.96 | 0.06 |
| 19 | Head: mean CTT acc | 1.879 | 0.071 |
| 20 | Left arm: max GUP av-x | 1.838 | 0.076 |
| 21 | Hip: max CTT av-y | 1.837 | 0.084 |
| 22 | Shoulder: mean CTT av-x | −1.805 | 0.083 |
| 23 | Right arm: median CTT av-x | −1.775 | 0.086 |
| 24 | Left leg: median GUP av-m | −1.775 | 0.086 |
| 25 | Left knee: median GUP vel | 1.763 | 0.091 |
| 26 | Right leg: mean GUP av-x | −1.742 | 0.091 |
| 27 | Spine mid: max CTT acc | 1.727 | 0.094 |
| 28 | Left hip: mean CTT ke | 1.702 | 0.098 |

(vel: velocity; acc: acceleration; pe: potential energy; ke: kinetic energy; sa: sagittal angle; av-x, av-y, av-z: angular velocity about x, y, or z axes).

Above examples demonstrate that using a motion capture system and wearable motion sensor may yield kinematic data that may correlate and determine important clinical outcomes such as unexpected healthcare encounters. As mentioned above, the kinematic features were based off of 25 anatomical sites that include head, arms, spine, hips, knees, and feet. Five kinematic features of the chair-to-table exam correlated with unexpected hospital visits. The anatomic sites that were statistically significant were left (non-pivoting) knee and hip, as well as the spine base. The spine base velocity may reflect the movement of a majority of the patient's mass that is not subject to high variability such as the distal hands or feet.

The association between high physical activity level and kinematic features may revolve around leg, knee, hip, and back movement. Similarly to above, these areas of the body intuitively may carry the majority of a patient's mass and lower extremities generally may be a more predictive measurement of a patient's overall physical activity. This was supported by the calculated kinematic features (Table 2).

The mean hip and minimum left leg angular velocities about the x-axis during get-up-and-go may be the two best differentiators of HALPA groups (FIG. 8), and both these angular velocities may be greater for patients with higher physical activity compared to patients grouped in the low activity group. Mean sagittal angle during CTT may generally be lower for patients with higher physical activity, which may be due to the increased ability of more active patients to crouch lower in the seated position before standing up and after reaching the medical table.

Identifying high-risk patients may be one approach to reduce costly preventable hospitalizations in cancer patients. Other approaches may include enhancing access and care coordination, standardize clinical pathways for symptom management, availability of urgent cancer care, and early use of palliative care.

Patient performance and physical activity may reliably be quantified using camera based kinematic analysis. Modern sensor technology may make such as assessment rapid and low cost. Such systems that quantifies what the physician sees during a clinic examination may have the potential to harmonize findings among different physicians, specialists, researchers and families who all rely on a uniform assessment of patient fitness for receiving difficult cancer treatments.

Although the present technology has been described in detail for the purpose of illustration based on what is currently considered to be the most practical, it is to be understood that such detail is solely for that purpose and that the technology is not limited to the disclosed embodiments, but, on the contrary, is intended to cover modifications and equivalent arrangements that are within the spirit and scope of the appended claims. For example, it is to be understood that the present technology contemplates that, to the extent possible, one or more features of any embodiment can be combined with one or more features of any other embodiment.

Exemplary features of the system and the method of this disclosure, which may be used for determining quantitative health-related performance status of a patient, may further be disclosed through the following claims:

1. A system for determining a quantitative health-related performance status of a patient, the system comprising:
at least one sensor; and
at least one processor;
wherein the system is configured to generate at least one output signal conveying physical activity information corresponding to physical activity of the patient, or spatial position information corresponding to at least one spatial position of an anatomical site of the patient while the patient performs a movement;
wherein the system is configured to determine at least one physical activity parameter or at least one kinematic parameter based on the at least one output signal; and
wherein the system is further configured to determine a quantitative health-related performance score of the patient based on the physical activity parameter or the kinematic parameter.

2. The system of any of claim 1, wherein the system is further configured to determine whether the patient will need unplanned medical care during a therapy based on the quantitative health-related performance score.

3. The system of claim 1, wherein the movement performed by the patient is a prescribed movement.

4. The system of claim 1, wherein the system further comprises an information conveying device that conveys information to a human user, wherein the conveyed information is related to the quantitative health-related performance score and/or the determination of whether the patient will need unplanned medical care.

5. (canceled)

6. (canceled)

7. The system of claim 1, wherein the at least one sensor comprises a body position sensor and/or a physical activity sensor.

8. The system of claim 1, wherein the system further comprises a system comprising an image recording device.

9. The system of claim 1, wherein the system further comprises a system comprising a 3D motion capture device.

10. The system of claim 1, wherein the system further comprises a system comprising a 3D motion capture device, and wherein the 3D motion capture device comprises an image recording device, a time-of-flight measurement device, a heat sensor, and a combination thereof.

11. The system of claim 1, wherein the system further comprises a system comprising a ToF sensor.

12. The system of claim 1, wherein the at least one sensor generates the at least one output signal conveying physical activity information corresponding to physical activity of the patient, or the spatial position information corresponding to at least one spatial position of an anatomical site of the patient while the patient performs a movement.

13. The system of claim 1, wherein the at least one processor determines the at least one physical activity parameter or at least one kinematic parameter based on the at least one output signal.

14. The system of claim 1, wherein the at least one processor determines the quantitative health-related performance score of the patient based on the physical activity parameter or the kinematic parameter.

15. The system of claim 1, wherein the at least one processor determines whether the patient will need unplanned medical care during a therapy based on the quantitative health-related performance score.

16. The system of claim 1, wherein the at least one sensor comprises a body position sensor, a wearable physical activity tracker, a balance, a system comprising an image recording device, a display, or a combination thereof.

17. The system of claim 1, wherein the at least one sensor comprises a wrist worn motion sensor.

18. The system of claim 1, wherein the system comprises a mobile phone.

19. (canceled)

20. The system of claim 1, wherein the anatomical site comprises a center of mass of the patient's body or a center of mass of the patient's body part.

21. (canceled)

22. (canceled)

23. (canceled)

24. The system of claim 1, wherein the spatial position information comprises visual information representing the patient's body, the patient's weight, the patient's height, the patient's body-mass-index (BMI), or a combination thereof.

25. The system of claim 1, wherein the system is configured to generate spatial position information of at least two spatial positions, determine at least one kinematic parameter for each spatial position, compare these kinematic parameters with each other, and determine whether the patient will need unplanned medical care during a therapy and/or during a future period of time based on this comparison.

26. The system of claim 1, wherein the system is further configured to generate spatial position information of a reference site unrelated to the patient; and determine whether the patient will need unplanned medical care based on the kinematic parameter determined by using the prescribed movement site relative to the reference site.

27. The system of claim 26, wherein the reference site comprises an exam table, a patient bed, a computer, or a combination thereof.

28. The system of claim 1, wherein the at least one kinematic parameter of the at least one spatial position comprises velocity, acceleration, specific kinetic energy, specific potential energy, sagittal angle, angular velocity, or a combination thereof.

29. The system of claim 1, wherein the at least one kinematic parameter comprises acceleration of the patient's non-pivoting knee, acceleration of the patient's non-pivoting hip, angular velocity of the patient's hip, angular velocity of the patient's non-pivoting leg, or a combination thereof.

30. The system of claim 1, wherein the at least one kinematic parameter comprises chair-to-table acceleration of the patient's non-pivoting knee, chair-to-table acceleration of the patient's non-pivoting hip, chair-to-table angular velocity of the patient's hip, chair-to-table angular velocity of the patient's non-pivoting leg, or a combination thereof.

31. The system of claim 1, wherein the determination of the at least one kinematic parameter comprises:
determining spatial position vectors for the at least one spatial position; and
determining acceleration of the at least one spatial position based on the spatial position vectors using a mean-value theorem;
wherein:
the spatial position vectors comprise three-dimensional time series generated for given positions of the at least one spatial position at a given time point during the prescribed movement; and
the acceleration of the at least one spatial position is determined using the mean-value theorem based on the spatial position vectors of the spatial position of the center of mass.

32. The system of claim 1, wherein the determination of the at least one kinematic parameter is indicative of the movement of the patient during a prescribed movement based on the spatial position information.

33. (canceled)

34. The system of claim 32, wherein the prescribed movement comprises movement associated with a chair to table (CTT) exam and/or a get up and walk (GUP) exam.

35. The system of claim 1, wherein the at least one physical activity parameter comprises at least one metabolic equivalent of task (MET).

36. The system of claim 1, wherein the determination of the at least one physical activity parameter is indicative of the physical activity of the patient.

37. The system of claim 25, wherein the determination of whether the patient will need unplanned medical care during therapy and/or the future period of time is based on the kinematic parameter; and/or the at least one physical activity of the patient.

38. The system of claim 1, wherein the system is further configured to categorize the patient as either likely to need unplanned medical care or unlikely to need unplanned medical care during the therapy, wherein the categorization comprises determining Eastern Cooperative Oncology Group (ECOG) scores.

39. The system of claim 4, wherein the determining whether the patient will need unplanned medical care during the therapy comprises comparing the acceleration of the spatial position of the center of mass to an acceleration threshold, and determining the patient will need unplanned medical care during the therapy responsive to a breach of the acceleration threshold.

40. The system of claim 4, wherein the determining whether the patient will need unplanned medical care comprises comparing a spine base acceleration time series to a corresponding baseline, determining a distance between the spine base acceleration time series and the corresponding baseline using Euclidean metric dynamic time warping (DTW), which assigns a distance of zero for completely identical series and larger distances for more dissimilar series, and determining the patient will need unplanned medical care during the therapy responsive to a breach of one or more DTW distance thresholds.

41. The system of claim 4, wherein unplanned medical care comprises a medical care unrelated to the therapy, an unscheduled medical care, a non-routine medical care, an emergency medical care, or a combination thereof.

42. The system of claim 1, wherein the system is further configured to facilitate adjustment of the therapy based on the determination of whether the patient will need unplanned medical care during the therapy.

43. The system of claim 4, wherein the determination of whether the patient will need unplanned medical care during the therapy is indicative of a future reaction of the patient to planned therapeutic intervention.

44. The system of claim 4; wherein the determination of whether the patient will need unplanned medical care during the therapy is indicative of a future reaction of the patient to planned therapeutic intervention; and wherein the target therapeutic intervention comprises chemotherapy, radiation therapy, immune therapy, hormone therapy, or a combination thereof.

45. The system of claim 4, wherein the determination of whether the patient will need unplanned medical care during the therapy is indicative of a future reaction of the patient to chemotherapy and/or radiation during the therapy.

46. The system of claim 4, wherein the determining whether the patient will need unplanned medical care during the therapy comprises determining whether the patient will need unplanned medical care during a future period of time that corresponds to at least one therapy treatment received by the patient.

47. (canceled)

48. The system of claim 4, wherein the determining whether the patient will need unplanned medical care during the therapy comprises:
determining a likelihood the patient will need unplanned medical care; and
categorizing the patient into two or more groups based on the likelihood;
wherein:
the likelihood comprises a numerical value on a continuous scale; and
the likelihood is inversely correlated to the acceleration of the spatial position of the center of mass.

49. The system of claim 2, wherein the therapy comprises a cancer therapy.

50. (canceled)

51. A quantitative health assessment method for quantitative determination of health-related performance or quality of life of a patient, the method comprising:
using a quantitative health assessment system of claim 1; and
determining whether the patient will need unplanned medical care during a therapy and/or during a future period of time.

52. The method of claim 51, wherein the patient is a clinical trial subject.

53. The method of claim 51, wherein the method further comprises deciding whether to continue, stop, or modify the therapy.

54. The method of claim 51, wherein the method further comprises deciding whether to stop or modify the therapy.

55. The method of claim 51, wherein the method further comprises deciding whether to stop the therapy.

56. The method of claim 51, wherein the patient is a clinical trial subject; and wherein (i) the method further comprises deciding whether to enroll the patient in a clinical trial or (ii) the method further comprises deciding whether to terminate the subject's participation in a clinical trial.

57. (canceled)

58. The method of claim 51, wherein the therapy is a therapy related to a clinical trial; and wherein the method further comprises deciding whether to stop or modify the clinical trial.

59. The method of claim 51, wherein the therapy is a therapy related to a clinical trial; and wherein the method further comprises determining a total number of unplanned medical care occurred during the clinical trial; and using this total number in deciding whether the therapy provided a better/improved health-related quality of life to the patient as compared to another therapy.
